(19) United States
(12) Patent Application Publication
(10) Pub. No.: US 2024/0428958 A1
(43) Pub. Date: Dec. 26, 2024

(54) METHODS AND SYSTEMS FOR IMPLEMENTING GENERATIVE AI IN MULTI-CHANNEL SERVICE PLATFORMS

(71) Applicant: Polyview Health, Inc., San Diego, CA (US)

(72) Inventors: Jason Hwang, Henderson, NV (US); Gregory Deocampo, Safety Harbor, FL (US); Dimitri Arges, Henderson, NV (US)

(21) Appl. No.: 18/748,172

(22) Filed: Jun. 20, 2024

Related U.S. Application Data
(60) Provisional application No. 63/509,910, filed on Jun. 23, 2023.
(60) Provisional application No. 63/509,973, filed on Jun. 23, 2023.
(60) Provisional application No. 63/510,006, filed on Jun. 23, 2023.
(60) Provisional application No. 63/510,019, filed on Jun. 23, 2023.
(60) Provisional application No. 63/510,608, filed on Jun. 27, 2023.
(60) Provisional application No. 63/604,930, filed on Dec. 1, 2023.

(51) Int. Cl. G16H 80/00; H04L 65/1083; H04L 67/306

(52) U.S. Cl. CPC G16H 80/00; H04L 65/1083; H04L 67/306

(57) ABSTRACT

The method and system described herein may include receiving a request to conduct a communication session between one or more user devices. A first user device of the one or more user devices may be a healthcare provider. The method may further comprise receiving user profile data, wherein the user profile data pertains to at least one or the one or more user devices of the communication session. Additionally, the method may detect communications between the one or more user devices over a duration of the communication session. The method may further comprise generating an object using a machine-learning model and presenting the object to at least one of the one or more user devices.

### CROSS-REFERENCE TO RELATED APPLICATIONS

The present patent application claims the benefit of priority to U.S. Provisional Patent Applications 63/509,910, 63/509,973, 63/510,006, and 63/510,019, all of which were filed Jun. 23, 2023; U.S. Provisional Patent Application 63/510,608, filed Jun. 27, 2023; and U.S. Provisional Patent Application 63/604,930, filed Dec. 1, 2023, which are all incorporated herein by reference in their entirety for all purposes.

### TECHNICAL FIELD

This disclosure relates generally to communication sessions. More specifically, this disclosure relates to generating a communication session environment incorporated with machine-learning capabilities customized according to one or more user devices on a multi-channel service platform.

### BACKGROUND

Telehealth can be conducted between a patient and a healthcare provider over a variety of communication channels. These telehealth calls are often facilitated in a standard format or based on third-party's pre-existing systems (e.g., Zoom, Microsoft Teams, Facetime, etc.). Because of the basic nature of current telehealth calls, the calls themselves may not be as beneficial or as efficient as they could be for both the patient and the healthcare provider. For example, it might be more difficult for a patient or healthcare provider to focus on the telehealth call or provide relevant information. As another example, telehealth calls often cause the patient to feel disengaged from the appointment and the healthcare provider, thus creating an environment where the patient might not trust the healthcare provider's advice or might not feel comfortable asking questions.

### SUMMARY

Methods and systems are described herein for generating a communication session environment on a multi-channel service platform. The method comprises: receiving a request, via a communication network, to establish a communication session between one or more user devices, wherein a first user device of the one or more user devices is a healthcare provider; receiving user profile data, wherein the user profile data pertains to at least one of the one or more user devices of the communication session; generating a communication session environment configured to manage the communication session, wherein the communication session environment is customized based on the user profile data; receiving from the first user device a communication associated with the user profile data; and generating, within the communication session environment, an object using the communication, wherein the object is contextually related to a purpose in which the communication session is established.

Systems are described herein for implementing generative AI in a multi-channel service platform. The systems include one or more processors and a non-transitory computer-readable storage medium storing instructions that, when executing by the one or more processors, cause the one or more processors to perform any of the methods as previously described.

A non-transitory computer-readable medium described herein may store instructions which, when executed by one or more processors, cause the one or more processors to perform any of the methods as previously described.

These illustrative examples are mentioned not to limit or define the disclosure, but to aid understanding thereof. Additional embodiments are discussed in the Detailed Description, and further description is provided there.

### BRIEF DESCRIPTION OF THE DRAWINGS

Features, instances, and advantages of the present disclosure are better understood when the following Detailed Description is read with reference to the accompanying drawings.

FIG. 1 illustrates a block diagram of example communication network configured to facilitate secured communication sessions between users according to aspects of the present disclosure.

FIG. 2A illustrates an example patient user interface of the communication network configured for a particular communication session according to aspects of the present disclosure.

FIG. 2B illustrates an example provider user interface of the communication network configured for a particular communication session according to aspects of the present disclosure.

FIG. 2C illustrates an example temporary environment user interface of the communication network configured for a particular user device according to aspects of the present disclosure.

FIG. 3 illustrates an example collaborative window containing AI-generated elements according to aspects of the present disclosure.

FIG. 4 illustrates a flowchart of an example process for dynamically generating objects within real time collaborative windows according to aspects of the present disclosure.

FIG. 5 illustrates an example computing device according to aspects of the present disclosure.

### DETAILED DESCRIPTION

Various instances of the disclosure are discussed in detail below. While specific implementations are discussed, it should be understood that this is done for illustration purposes only. A person skilled in the relevant art will recognize that other components and configurations may be used without parting from the spirit and scope of the disclosure.

Methods and systems are described herein for providing a communication session environment within a multi-channel service platform. In some instances, a communication session may be conducted between a first user device associated with a patient and a second user device associated with a healthcare provider as part of a telehealth session. The healthcare provider may be a doctor, nurse, therapist, and/or another healthcare professional. The patient may be a current patient of the healthcare provider or may be a new patient. One or more other user devices may also be connected to the communication session. The one or more other user devices may be associated with other users (e.g., such as additional patients, users associated with the patient such as a nurse, additional healthcare providers, etc.). For example, an adult child of an elderly parent may participate in a communication session with the elderly parent and a healthcare provider of the elderly parent.

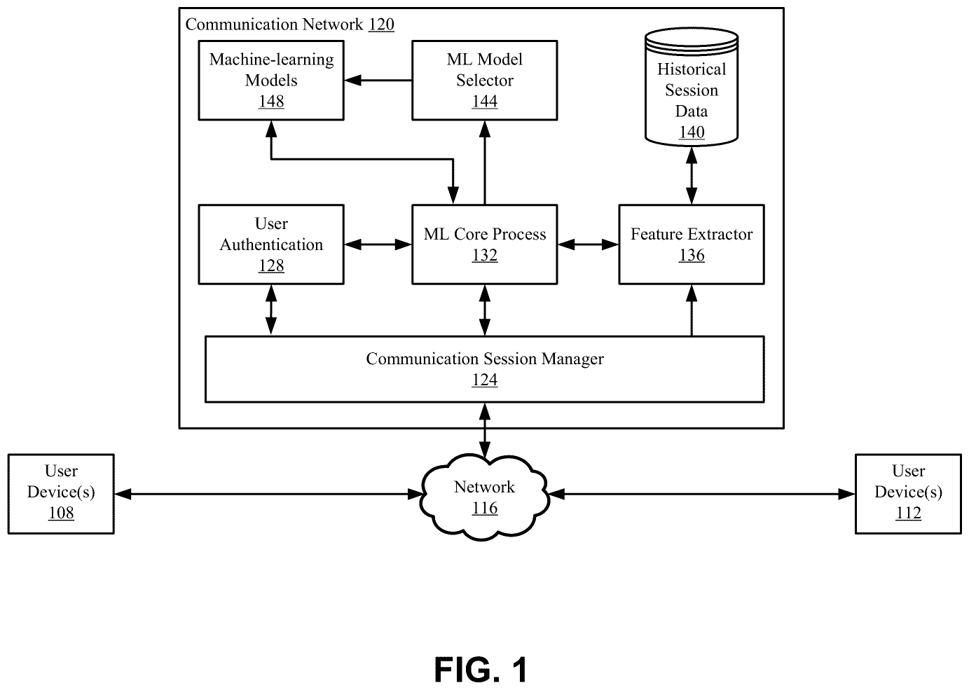

The first user device may transmit a communication session request to a communication network (e.g., communication network 120 of FIG. 1, an application, network, etc.). The communication session may be facilitated over one or more communication channels (e.g., telephone or other audio-based communication channels, video or other audiovisual-based communication session, text such as text messaging or instant messaging, combinations thereof, or the like. In some instances, the communication channel may be asynchronous or communications may be transmitted over the communication channel asynchronously. For example, the first user device may communicate over an audio channel and lack a capability of receiving or processing data received over a video channel (e.g., due to permissions or other security settings, processing capabilities, available resources, available bandwidth, etc.). The second user device may communicate over both audio and video, etc. such that the first user device may receive audio from the second user device and the second user device may receive both audio and video from the first user device.

The communication session request may include a set of communication session parameters. The set of communication session parameters may include, but are not limited to, a quantity of user devices that are authorized to connect to the communication session, an identification of the user devices or the users thereof (e.g., such a device identifier, Internet Protocol address, email address, phone number, username, a user identifier, combinations thereof, or the like), a length of the communication session, an identification of one or more communication channels authorized for the communication session (e.g., such as, audio, video, audiovisual, text messaging, email, instant messaging, combinations thereof, or the like), video settings, sound settings, collaborative environment parameters, privacy and/or encryption parameters, artificial intelligence accessibility, date and time, etc. The set of communication session parameters may be modified at any time prior to and/or during the communication session.

In an illustrative example, the set of communication session parameters may include an identification of one or more other user devices, which may include the second user device. The one or more other user devices may be associated with a healthcare provider, another patient, someone associated with the patient (e.g., such as a social worker, nurse, aide, etc., a third party, etc.) The first user device may identify the one or more other user devices as user devices to be invited to connect to the communication session.

The communication network may transmit a notification to the one or more other user devices invited to the communication session using information of the set of communication session parameters. The notification may include a representation of one or more communication session parameters of the set of communication session parameters. The one or more other user devices may accept or decline the communication session. A user device of the one or more second user devices may request a modification to one or more of the communication session parameters (e.g., such the date/time of the communication session, identification of user devices authorized to access the communication session, etc.). The communication network may receive a response from the one or more other user devices (or other user devices associated with the communication request). The one or more other user devices may modify a response of accepting or declining the communication session request (e.g., changing an acceptance to a decline, changing a decline to an acceptance, etc.). The communication session parameters may determine an action if no response is received from a user device of the one or more other user devices. In some instances, the communication network may indicate that a lack of response from the user device may be an acceptance. In other instances, the communication network may indicate that a lack of response from the user device may be a decline.

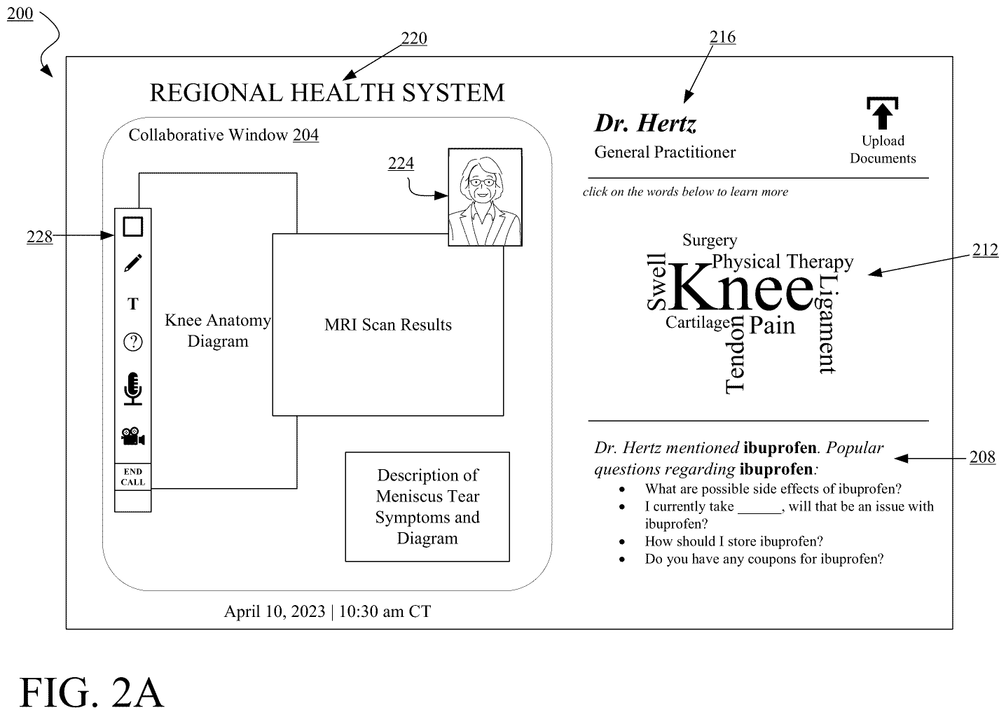

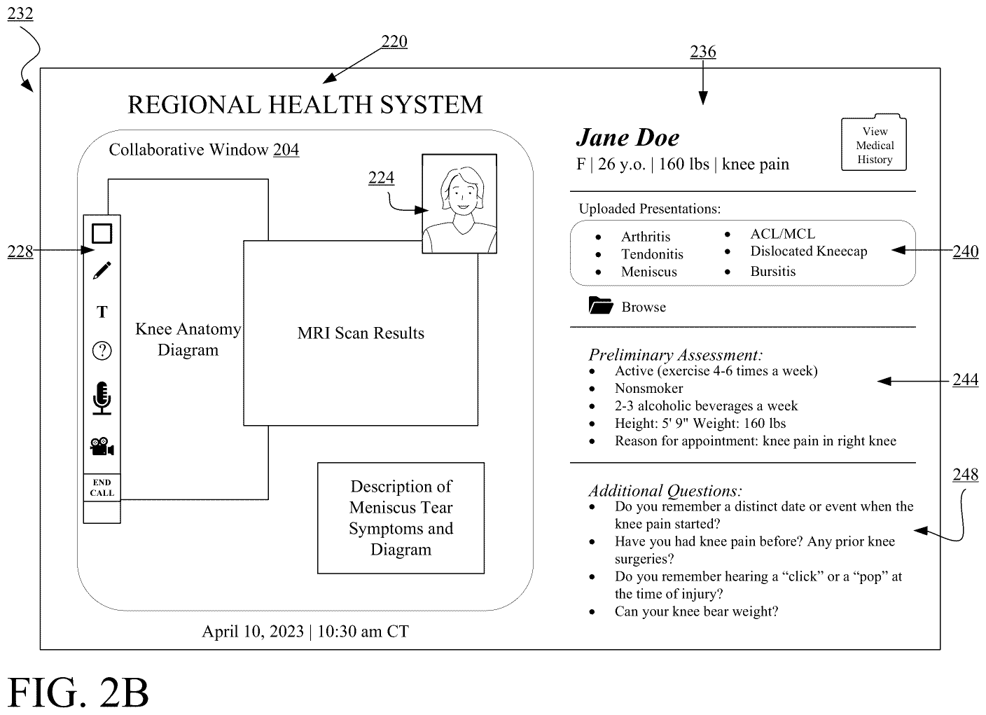

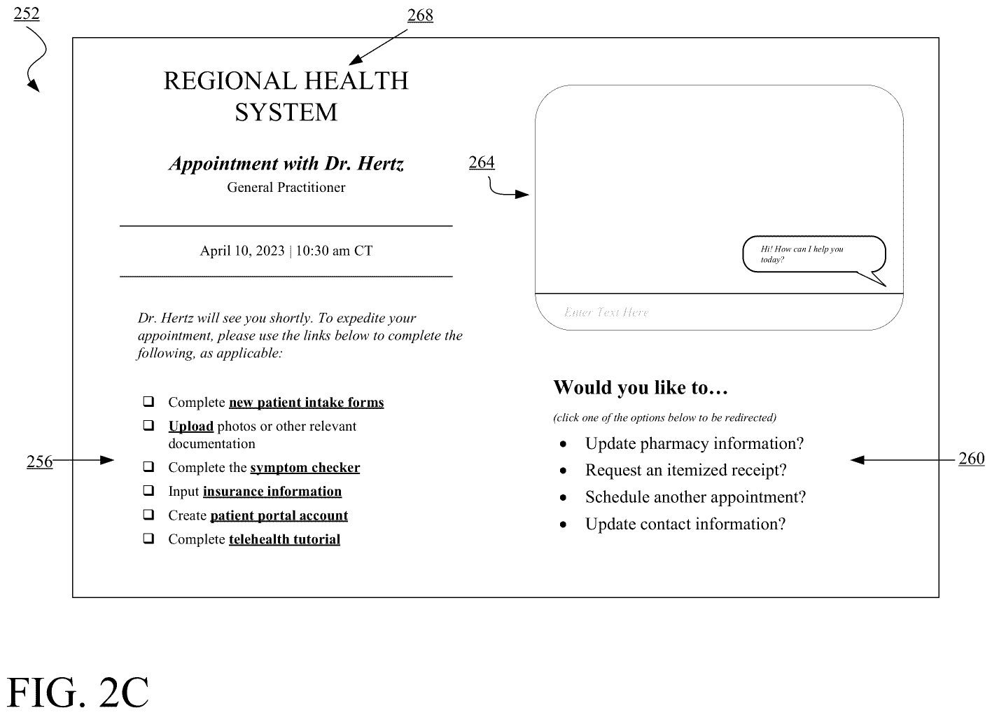

In some examples, the communication network may facilitate access to a telehealth environment. The telehealth environment may be operated by the communication network, the healthcare provider, an entity associated with the healthcare provider, etc. In some instances, the telehealth environment may be an application, website, or like configured to store and provide information associated with the first user device and facilitate the communication session. In other instances, the telehealth environment may be a virtualized environment (e.g., such as, but not limited to a virtual machine, secure processing environment, or the like) configured to execute processes and/or applications, store and/or provide information associated with the user device, facilitate communications between two or more devices, etc. The telehealth environment may include a sub-environment for facilitating the communication session (e.g., as shown FIG. 2A).

The telehealth environment may include a linked post-session environment. The post-session environment may include an identification of previous communication sessions associated with the first user device. The telehealth environment may include an application or plugin configured to replay a previous communication session. In some instances, the telehealth environment may include a link to an environment associated with the healthcare provider where the previous communication session may be stored. The first user device may access the link to connect to the environment associated with the healthcare provider to access the previous communication session. Alternatively, the communication network may embed the previous communication session hosted by the environment associated with the healthcare provider within the telehealth environment to enable the first user device to access the previous communication session without leaving the telehealth environment. The post-session environment may also include resources presented by user devices during communications sessions, resources provided by user devices before or after a communication session (e.g., charts, explanations, prescriptions, test results, notes, instructions, etc.), access to artificial intelligence (AI) resources (e.g., natural language processors for speech-to-text, text-to-speech, translation, classification, etc.; large language models or other generative models for automated communication and information generation, etc.), other resources, administrative links (e.g., pay bill, schedule appointment, etc.), transcripts of prior communication sessions, potential questions for the healthcare provider, etc.

The communication network may provide access to the communication session when a current time corresponds to a time identified by a communication session parameter. The communication network may establish the communication session through the telehealth environment using a sub-environment configured to enable communications over one or more communications channels identified by a communication session parameter of the set of communication session parameters. Alternatively, the communication network may establish the communication session using a third-party environment (e.g., video conferencing application or environment provided by an entity other than the communication network, etc.). The first user device and the one or more other user devices may access the communication network and/or the communication session using via the Internet, local or wide area network, a cellular network, etc.

In some examples, the communication network may include an access sequence restriction that defines an order in which user devices may access the communication session. The access sequence restriction may comprise an access sequence order, wherein the access sequence order may position the first user device and the one or more other user devices sequentially. The communication network may connect the one or more user devices positioned lower in the access sequence order to a temporary environment (e.g., a virtual waiting room, etc.) until the one or more user devices positioned higher in the access sequence order connect to the communication session. For example, the communication network may indicate that the first user device cannot join the communication session until the second user device connects to the communication session.

The temporary environment may enable user devices to access information associated with the communication session and transmit information to the communication network or healthcare provider (e.g., intake healthcare forms, medical history, insurance information, etc.). The information associated with the communication session can include a tutorial or other introductory information that provides information on features of the communication network and communication session as well as how to access the features, information associated with the healthcare provider (e.g., such as names and/or address of healthcare practitioners, services offered by the healthcare provider, branches of medicine practiced by the healthcare provider, affiliated healthcare providers, emergency information, etc.).

The temporary environment may include one or more automated services (e.g., chatbots, large language models, generative models, natural language understanding models, image and/or audio processors, etc.) configured to interact with the user devices of the temporary environment over various communication channels. The one or more automated services may be configured to communicate using natural language communications in a language selected by a user device communicating with the automated services. The user devices may ask questions related to the communication session or the purpose for the communication session, request information associated with the healthcare provider or communication network, etc. and receive responses from the automated service in a natural language format and in the select language. In some instances, the second user device may be presented with the questions or requests for information and provide the response in place of or in addition to the automated service. The communication network may mark responses generated by an automated service different from responses generated by the healthcare provider to enable a user to determine an origin of a particular response.

In some instances, the resources available in the temporary environment are determined by an associated health system. The associated health system may be the hospital system affiliated with the clinic, office, hospital, etc. where the healthcare provider conducts healthcare services to patients. For example, the resources available in the temporary environment may be a map of the hospital, operating hours of the clinic and/or hospital, a link to a webpage associated with the associated health system, or any other resource that may be specific to the associated health system. The healthcare provider may also select the resources available in the temporary environment. As another example, if the healthcare provider receives information indicating that the patient has a sore throat, the healthcare provider may generate a symptom checker for the patient to complete within the temporary environment, provide diagnostic information associated with sore throats and possible causes, provide treatment information, etc.

The communication session may be facilitated by a communication session environment within the telehealth environment. The communication session environment may include a user interface generated by the communication network to facilitate the communication session. The user interface may be different for each user device or class of user devices connected to the communication session. For example, user devices associated with healthcare providers may be presented with a different user interface than user devices associated with patients. The user interface of the first user device may appear similar to the user interface illustrated in FIG. 2A, while the user interface of the second user device may appear similar to the user interface illustrated in FIG. 2B. The user interface may enable presentation of information associated with the communication session during the communication session. The communication network may enable any user device connected to the communication session to share content (e.g., read and/or write to the user interface, etc.) based upon the communication session parameters. For example, the communication session parameters may enable the second user device to share content and/or read/write to the user interface while the first user device may only display information provided by the healthcare provider.

The user interface may display resources provided by user devices connected to the communication session and/or resources provided by one or more machine-learning models. For example, the communication network may utilize a natural language processing (“NLP”) machine-learning model to present a live transcript of the communication session to a user device operated by a user that may be hearing impaired. The resources may be determined by preferences of the associated health system and/or preferences of the first user device. The resources provided through the user interface may also be determined by the second user device. For example, the second user device connected to the communication session may limit access to an AI-assisted question generator from the user interface of the first user device until the last ten minutes of the communication session.

In some examples, the communication network may provide user devices associated with healthcare providers access to additional resources pertaining to healthcare services. For example, the healthcare provider may have access to patient health records, test results, presentations associated with the communication session or the first user device (e.g., charts, graphs, images, videos, etc.). The first user device may have access to other resources. For example, the first user device may be presented with a set of pre-generated questions generated by a machine-learning model (e.g., such an NLP machine-learning model, large language model, etc.) or pre-generated based on an association with an issue for which the communication session was requested.

Within the communication session environment for the first user device and the one or more second user devices, there may be a collaborative window synchronized amongst all user devices. The collaborative window may be displayed by the user interface of a user device. The collaborative window may be updated in real-time and may enable any user device connected to the communication session to edit the collaborative window according to the collaborative session parameters. The edits may be shared in real-time to user devices connected to the communication session. The collaborative window may be associated with one or more settings configured within the communication session parameters. For example, the communication session parameters may grant permissions to one or more user devices permitted to edit/share content on the collaborative window. The communication session parameters may also determine what types of content may be shared on the collaborative window (e.g., videos, photos, diagrams, presentations, text, etc.). The collaborative window may contain a dynamic video view, wherein the video feed of a user's face from the user device currently outputting speech or sound data may be transmitted to the user devices connected to the communication session via the collaborative window.

Over the duration of the communication session, the communication network may utilize the communication (e.g., audio, visual, etc.) between the user devices connected to the communication session and generate a data package containing a recording of the communication session (e.g., containing audio, visual, etc. content outputted over the duration of the communication session) and data generated by other AI-assisted resources. After the communication session is concluded, the user devices connected to the communication session may receive a notification (e.g., via email, telehealth environment notification, text message, etc.) indicating the data package is available for examination. The data package accessible to each user device may differ based on the user device. For example, the data package of each user device may present a representation of the communication session that was presented to that device during the communication session. The first user device may access a data package with a representation of the communication session that was presented to the first user during the communication session. In some instances, the data package includes additional data derived from the communication session such as additional information about objects presented in the user interface (including the collaborative environment, etc.), statements made by the healthcare provider or patient, etc. The additional data may be generated automatically (e.g., by one or more of the AI resources or automated services previously described), generated by the healthcare provider, generated by the patient, or another entity that was connected to the communication session.

FIG. 1 illustrates a block diagram of example communication network configured to facilitate secured communication sessions between users according to aspects of the present disclosure. Communication network 120 may facilitate communication sessions between a first user device operated by a patient (e.g., user device 108) and a second user device operated by a healthcare provider (e.g., user device 112). Communication network 120 may enable one or more other devices associated with the first user device (e.g., such as nurses, aides, social workers, parents, adult children, etc.) and/or the second user device (e.g., nurses, assistants, other doctors, administrators, etc.) to connect to the communication session.

Communication network 120 may include one or more processing devices (e.g., computing devices, mobile devices, servers, databases, etc.) configured to operate together to provide the services of communication network 120. The one or more processing devices may operate with a same local network (e.g., such as a local area network, wide area network, mesh network, etc.) or may be distributed processing devices (e.g., such a cloud network, distributed processing network, or the like). User device 108 and user device 112 may connect to communication network 120 directly or through one or more intermediary networks 116 (e.g., such as the Internet, virtual private networks, etc.).

The first user device or the second user device may request a new communication session using communication session manager 124. The request may include an identification of one or more user devices are authorized to connect to the communication session. The request may also include other parameters such as user profile data (associated with the first user device or the second user device, such as, but not limited to an identification of a user of the user profile, a user identifier, user devices operated by the user), a purpose for establishing the communication session, a start time of the communication session, an expected duration of the communication session, settings of the communication session (e.g., audio channel settings, video channel settings, collaborative window settings, wrapper settings, etc.), combinations thereof, or the like.

Communication session manager 124 may then instantiate a new communication session for the first user device and/or the second user device. The new communication session may include one or more environments for the user devices connected to the communication session. The environment may include user interfaces, wrappers, resources, application programming interfaces, etc. configured to extend the functionality of the communication session. The communication session manager 124 using ML core process 132 may provision one or more machine-learning models to enable any of the extended functionality. The one or more machine-learning models may be configured to provide natural language processing (e.g., such as a large language model, bi-directional transformers, zero/few shot learners, deep neural networks, etc.), content generation (e.g., using large language models, deep neural networks, generative adversarial networks, etc.), single variate or multivariate classifiers (e.g., k-nearest neighbors, random forest, logarithmic regression, decision trees, support vector machines, gradient decent, etc.), image processing (e.g., using deep neural networks, convolutional neural networks, etc.), sequenced data processors (e.g., such as recurrent neural networks, etc. capable of processing datasets organized according to a taxonomic sequence), and/or the like. The one or more machine-learning models may be configured to process natural language communications (e.g., such as gestures, verbal, textual, etc.) to provide real-time translations and/or transcriptions, generate natural language communication capable of autonomous communication (e.g., a communication bot) or content generation (e.g., such generating natural language responses to requests for information, etc.), user authentication (e.g., to ensure users connected to the communication session are authorized to do so), and/or the like.

Communication session manager 124 may authenticate each user device that connects to the new communication session using user authentication 128. User authentication 128 may ensure that the user of a connected user device corresponds to an authorized user. To avoid exposing personal identifiable information or medical information, user authentication 128 may compare abstracted features associated with the user to corresponding abstracted features associated with an authorized user. In some instances, the abstracted features may include an abstracted representation of a username, password, token, public/private key, and/or the like. Communication session manager 124 may distribute passwords, tokens, public/private keys, and/or the like with an invitation to connect to the new communication session. In some instances, the abstracted features may include biometric features of a user of a user device to be authenticated such as physical features, vocal features, and/or the like. For example, using monocular depth estimation facial features can be extracted based on a relative distance of a representation of the facial feature (e.g., in a video frame) from the camera that captured the representation. The relative distances may be used to determine if the user a user device corresponds to a known, authenticated user by comparing the relative distances to stored relative distances.

User authentication 128 may obtain one or more video frames and/or one or more audio segments received from a user device to be authenticated. The one or more video frames may be generated using a camera of the user device and include a representation of a user of the user device. The audio segments may include a representation of a voice of the user. User authentication 128 may transmit a request to machine-learning (ML) core process 132 to process the video and/or audio. For example, using a first machine-learning model of machine-learning models 148, a depth map may be generated using a video frame including a representation of a user. The depth map may include a distance value for each pixel of the video frame corresponding to a predicted distance of a point in the environment represented by the pixel from the camera that captured the video frame. User authentication 128 may use the depth map to distinguish pixels corresponding to the user from pixels corresponding to the background (e.g., based on the pixels representing the user being generally closer than pixels representing the background). User authentication 128 may then determine relative differences in distances between one or more pixels to determine a relative depth of one or more facial features. The relive differences in distances may be abstracted features that may be used to determine whether the user represented by the video data is authenticated by comparing the abstracted features to abstracted features of an authenticated user.

Using a second machine-learning model, user authentication 128 may process audio segments including a representation of the user's voice. The second machine-learning model may process the audio segments to derive abstracted features associated with the audio segments, the second machine-learning model may be configured to identify pitch, tone, speech velocity, pause frequency and length, diction, accent, language, etc.) of the audio segment represented as a sequence of numerical values. The abstracted features can be compared to historical abstracted features of an authenticated user to determine if the user associated with the abstracted features is the same user as the user associated with the historical abstracted features.

Communication session manager 124 may pass communications extracted over the communication session to ML core process 132 process the communications using the one or more machine-learning models. ML core process 132 may monitor one or more machine-learning models configured to provide the services of the communication network. ML core process 132 may train new machine-learning models, retrain (or reinforce) existing machine-learning models, delete machine-learning models, and/or the like. Since ML core process 132 manages the operations of a variety of machine-learning models, each request to ML core process 132 may include an identification of a particular machine-learning model, a requested output, or the like to enable ML core process 132 to route the request to an appropriate machine-learning model or instantiate and train a new machine-learning model. Alternately, ML core process 132 may analyze data to be processed that is included in the request to select an appropriate machine-learning model configured to process data of that type.

If ML core process 132 cannot identify a trained machine-learning model configured to process the request, then ML core process 132 may instantiate and train one or more machine-learning models configured to process the request. Machine-learning models may be trained to process a particular input and/or generate a particular output. ML core process 132 may instantiate and train machine-learning models based on the particular data to be processed and/or the particular output requested. For example, user sentiment analysis (e.g., user intent, etc.) may be determined using a natural language processor and/or a classifier while image processing may be performed using a convolutional neural network.

ML core process 132 may select one or more machine-learning models based on characteristics of the data to be processed and/or the output expected. ML core process 132 may then use feature extractor 136 to generate training datasets for the new machine-learning models (e.g., other than those models configured to perform feature extraction such as some deep learning networks, etc.). Feature extractor 136 may define training dataset using historical session data 140. Historical session data 140 may store features from previous communication sessions. In some instances, the previous communication sessions may not involve the user of the first user device or the user of the second user device. Previous communication sessions may include manually and/or procedurally generated data generated for use in training machine-learning models. Historical session data 140 may not store any information associated with healthcare providers or patients. Alternatively, historical session data 140 may store features extracted from communication session involving the user of the first user device, the user of the second user device, and/or other patients and/or other healthcare providers.

Feature extractor 136 may extract features based on the type of model to be trained and the type of training to be performed (e.g., supervised, unsupervised, etc.) from historical session data 140. Feature extractor 136 may include a search function (e.g., such as procedural search, Boolean search, natural language search, large language model assisted search, or the like) to enable ML core process 132, an administrator, or the like to search for particular datasets within historical session data 140 to improve the data selection for the training datasets. Feature extractor 136 may aggregate the extracted features into one or more training datasets usable to train a respective machine-learning model of the one or more machine-learning models. The training datasets may include training datasets for training the machine-learning models, training datasets to validate an in-training or trained machine-learning model, training datasets to test a trained machine-learning model, and/or the like. The one or more training datasets may be passed to ML core process 132, which may manage the training process.

Feature extractor 136 may pass the one or more training datasets to ML core process 132 and ML core process 132 may initiate a training phase for the one or more machine-learning models. The one or more machine-learning models may be trained using supervised learning, unsupervised learning, self-supervised learning, or the like. The one or more machine-learning models may be trained for a predetermined time interval, a predetermined quantity of iterations, until one or more target accuracy metrics have exceeded a corresponding threshold function (e.g., accuracy, precision, area under the curve, logarithmic loss, F1 score, weighted human disagreement rate, cross entropy, mean absolute error, mean square error, etc.), user input, combinations thereof, or the like. Once trained, ML core process 132 may validate and/or test the trained machine-learning models using additional training datasets. The machine-learning models may also be trained at runtime using reinforcement learning.

Once the machine-learning models are trained, ML core process 132 may manage the operation of the one or more machine-learning models (stored with other machine-learning models in machine-learning models 148) during runtime. ML core process 132 may direct feature extractor 136 to define feature vectors from received data (e.g., such as the video data, audio segments from the first user device or the second user device, content of a collaborative window such as collaborative window 204, etc.). In some instances, ML core process 132 may facilitate generation of a feature vector each time there is a change in the communication channel (e.g., a change in video from a user device, an audio segment is transmitted over the communication channel, content is added or removed from a user interface of the communication session (e.g., such as patient interface 200, provider interface 232, temporary environment 252, or any other interface displayed to a user device), content is modified from a user interface of the communication session (e.g., such manipulating an image, etc.), a timestamp relative to a start time of the communication session, or the like. ML core process 132 may continually execute the one or more machine-learning models to generate corresponding outputs. ML core process 132 may evaluate the outputs to determine whether to manipulate a user interface of the communication session based on the output (e.g., post automatically generated content, modify a word wall weights or words, add/remove suggested questions, initiate an automated conversation with a bot, provide information associated with keywords spoken during the communication session, etc.).

For example, ML core process 132 may detect a new audio segment communicate over the communication session. ML core process 132 execute a machine-learning model (e.g., such as a recurrent neural network) to process the audio segment to determine the words within the audio segment (if any) and a sentiment (e.g., a predicted meaning of the individual words or the words as a whole). ML core process 132 may execute another machine-learning model (e.g., such as a classifier, a large language model and/or transformer, a generative adversarial network, etc.), to generate content corresponding to the words and/or sentiment that can be provided to a user device. For instance, the words may include “My knee is swollen and painful” with a sentiment of “symptoms.” The other machine-learning model may process the words and sentiment to generate content for patient interface 200 such as information about ailments associated with knee pain and knee swelling, home treatments that may alleviate symptoms and/or improve mobility, possible questions that can be asked to the healthcare provider, etc. ML core process 132 may also use the other machine-learning model to generate content for provider interface such as symptoms, suggested follow up questions regarding the degree of swelling or the intensity of the pain, links for additional information associated with knee pain or knee swelling, links associated with ailments associated with knee pain or knee swelling, etc.

ML core process may direct feature extractor 136 to define other feature vectors to process other data using machine-learning models of machine-learning models 148 in parallel with the aforementioned machine-learning models to provide other resources of communication network 120. ML core process 132 may execute any number of machine-learning models in parallel to provide the functionality of a communication session.

Communication session manager 124 may update the interfaces (e.g., patient interface 200, provider interface 232, temporary environment 252, and/or any other interface presented to user devices during a communication session) in real-time. Content may be received from a particular user device associated with a collaborative window (that may be viewable by all user devices connected to the communication session) via a drag and drop over the collaborative window, an upload link, or the like. Communication session manager 124 may process the received content into a format that can be embedded into the collaborative window and update the collaborative window (or the entire interface) enabling other user devices connected to the communication session to view the content in a same manner as provided by the particular user device. Communication session manager 124 may also receive outputs from machine-learning models 148 via ML core process 132 and determine whether to update the interfaces using the output and how to update the interfaces using the output (e.g., where to present generated content within the interface, what fonts and font sizes to present generated content, whether generated content is to expire after a time interval and be removed from the interface, whether the content is related to and should presented proximate to other content, etc.). Communication session manager 124 may continuously update and the interfaces to present a dynamic, collaborative interface to each user device through the communication session. When the communication session terminates, communication session manager 124 may store the interfaces with an identifier associated with the communication session for further processing and/or replay by a user device of the communication session.

FIG. 2A illustrates an example user interface of the communication network presented to user devices of a communication session according to aspects of the present disclosure.

Patient interface 200 may include one or more elements accessible by a first user device over the duration of a communication session. The first user device may be operated by a patient or an entity associated with the patient (e.g., nurse, assistant, aide, medical proxy, etc.). The one or more elements may contain content and/or links to content pertaining to the communication session. The one or more elements may be selected by a healthcare provider, a machine-learning algorithm, one or more other user devices, associated health system settings, any combination thereof, or the like. The one or more elements within patient interface 200 may be specific to a particular communication session. The one or more elements may be visual or may contain interactive portions, wherein the user device may “click” or “select” specific portions to view additional content or where the user device may provide information to one or more other devices connected to the communication session.

For example, collaborative window 204 may contain one or more objects. The objects may be selected automatically (e.g., by a machine-learning model, rules engine, etc.), by the second user device, and/or the first user device. For example, the second user device may present objects to the first user device pertaining to an ailment (e.g., symptoms, diagnosis, treatments, historical facts, lifestyle or dietary interactions, etc.). In another illustrative example, objects pertaining to test results and/or anatomic scans may be presented within collaborative window 204. The first user device and/or the second user device may review the shared content simultaneously in real-time. In some examples, the first user device may also generate presentations containing visual content and present them on collaborative window 204 (e.g., uploading images or video segments, drawing using a curser, transmitting alphanumeric text, etc.)

Toolbar 228 may contain tools for a user device to manipulate collaborative window 204. The tools may include, but are not limited to, a pencil tool, a shape tool, a text tool, a “mute” button, a “camera” button, and an “end call” button. The tools described herein may allow the user device to generate additional content for display to one or more user devices connected to the communication session. For example, the user device may use a “pencil” tool to circle a particular element of a set of content.

In some examples, camera view 224 may display a real-time camera view of the face of an individual (e.g., the patient or another user associated with a user device connected to the communication session) within collaborative window 204 if a user device is equipped with a camera. Camera view 224 may present an image of an individual operating the user device. If the user device is equipped with multiple cameras (e.g., a camera pointed away from the user and a camera pointed towards the user), then camera view 224 may display an output from one of the cameras according to settings on the user device. Camera view 224 may vary in size, depending on the size of the screen of the user device connected to the communication session, the content currently displayed in collaborative window 204, communication session parameters associated with the communication session, any combination thereof, or the like. Camera view 224 may change in size dynamically or may be changed by a user device connected to the communication session. Camera view 224 may be eliminated entirely from collaborative window 204, either temporarily or for the entirety of the duration of the respective communication session to enable access to an additional area of collaborative window 204, prevent a distraction, etc. Removing camera view 224 may include reducing a size of camera view 224 to a zero-by-zero pixel window or by causing the camera view 224 to become transparent. By maintaining the window in a reduced size and/or increased transparency may reduce the latency when bringing camera view 224 (e.g., resizing camera view 224 or reducing the transparency of camera view 224).

Camera view 224 may display a particular user associated with a user device connected to the communication session according to the user device currently outputting audio. For example, if three user devices are connected to a communication session, and the three user devices are associated with three respective individuals-a healthcare provider, a patient, and the patient's guardian-camera view 224 may display the healthcare provider while the healthcare provider is providing a diagnosis but may switch to a display of the patient's guardian if the patient's guardian begins to ask a question. In some examples, the communication network may selectively determine whether video transmitting from a user device should be displayed by camera view 224. For example, the communication session may determine not to display a video feed associated with the first user device, even if that first user device is outputting audio. For example, using the same communication session as above, camera view 224 of the second user device will not show the video feed of the healthcare provider, even if the healthcare provider is the one speaking. However, camera view 224 of the user devices associated with the patient and the patient's guardian may display the healthcare provider.

User interface wrapper 220 may comprise a graphic overlay that frames collaborative window 204 and displays content. User interface wrapper 220 may contain branding messaging, including, but not limited to, the associated health system logo, the communication network logo, the associated health clinic logo (e.g., a health clinic where an associated healthcare provider may actively practice), other content (e.g., advertisements, services provided by the healthcare provider or health clinic, etc.), and/or communication session appointment information (e.g., date, time, etc.). The content displayed on user interface wrapper 220 may cycle throughout the communication session. For example, the other content displayed on user interface wrapper 220 may change every ten seconds in a consistent loop, wherein the same five sets of content are shown in the same order repeatedly. In some instances, the other content may be selected based on the communication session (e.g., such as, but not limited to, based on words said by the healthcare provider, words said by the patient, information presented within collaborative window 204, combinations thereof, or the like).

In some examples, the user interface wrapper 220 may be extended to contain additional content, including healthcare provider information 216, key phrase panel 212, and question prompt 208. Healthcare provider information 216 may contain information pertaining to the current communication session. In some examples, it may include the name of the healthcare provider connected to the communication session and/or the medical practice area of the healthcare provider. In some examples, healthcare provider information 216 may include a location in which the healthcare provider is located (e.g., “Boston, Massachusetts,” an address of an office of the healthcare provider, etc.). Healthcare provider information 216 may also include an upload link. A user device and/or another user device connected to the communication session may upload documents, photos, documentation, surveys, etc. requested by the healthcare provider using the upload link. The user device may select whether information uploaded via the upload link is to be displayed within collaborative window 204 or sent directly to a particular user device connected to the communication session (e.g., the healthcare provider, etc.). The healthcare provider may request this information during or prior to the communication session. At time of upload, the communication network transmits the data in real time to a location accessible by the second user device, such as a network folder, cloud drive, shared storage drive, any combination thereof, or the like. The location accessible by the second user device may contain the entirety of the documentation provided by the user device, including, but not limited to, uploads from prior communication sessions.

Key phrase panel 212 may be generated by communication network 120 with a machine-learning algorithm and/or a natural language processing (“NLP”) machine-learning model. The NLP machine-learning model may receive transmitted audio data from the user devices connected to the communication session and identify one or more words and/or phrases appearing in the transmitted audio data. The NLP machine-learning model may transform the words and/or phrases into a graphic display, as shown in FIG. 2A, with the larger word/phrase size indicating more frequent appearances in the transmitted audio data. In some examples, the first user device may select and/or click the words and/or phrases comprising key phrase panel 212 to be redirected to a second user interface displaying additional information pertaining to the word and/or phrase. The second user interface may include a website, an internal document from the associated health system and/or the healthcare provider, a presentation, an application or service, any combination thereof, or the like. The other page may be generated by a machine-learning algorithm trained to select relevant materials for patients (e.g., such as a large language model, generative adversarial network, or the like). In some examples, the communication network may track the activity and/or interactions between the first user device and patient interface 200. The second user device may receive a notification including data pertaining to the activity and/or interactions of the first user device. For example, this data may be analyzed to indicate which topics the healthcare provider should revisit for the patient before the conclusion of the communication session or during a subsequent communication session. Key phrase panel 212 may be an abstract design, as shown in FIG. 2A, a list, or another visual representation of topics covered over the duration of the communication session.

In some instances, user interface wrapper 220 may contain question proposal 208. Question proposal 208 may utilize a similar NLP machine-learning system as key phrase panel 212. The NLP system may receive transmitted audio data from the user devices connected to the communication session and identify one or more words appearing in the transmitted audio data. A machine-learning algorithm may generate a set of questions directed towards the one or more words. The set of questions may be presented on the user device and the patient may select questions to ask the healthcare provider. The machine-learning algorithm may reference historical communication sessions between the one or more user devices connected to the communication session and/or all historical communication sessions conducted via communication network 120.

At the conclusion of the communication session, patient interface 200 may automatically close and the communication network may automatically redirect the user device to another location within the communication network or other connected webpage. Collaborative window 204, key phrase panel 212, question proposal 208, and/or other content displayed on user interface wrapper 220 may be made accessible to the one or more user devices connected to the communication session after the conclusion of the communication session via a transmitted link, a patient portal, the telehealth environment, an application or service, a webpage, any combination thereof, or the like.

FIG. 2B illustrates an example user interface of the communication network presented to user devices of a communication session according to aspects of the present disclosure.

Provider interface 232 may contain one or more elements accessible by a second user device over the duration of a communication session. The second user device may be operated by a healthcare provider. The one or more elements may contain content and/or links to content pertaining to the communication session. Provider interface 232 may differ from a user interface associated with a different user device (e.g., patient interface 200 described in FIG. 2A). The one or more elements may be selected by the healthcare provider, a machine-learning algorithm, one or more other user devices, associated health system settings, any combination thereof, or the like. The one or more elements within provider interface 232 may be specific to the communication session. The one or more elements may be visual or may contain interactive elements, wherein the user device may “click” or “select” specific elements to view additional content or where the second user device may provide information one or more other devices connected to the communication session. Provider interface 232 may include elements previously described in FIG. 2A (e.g., patient interface 200) including, but not limited to, collaborative window 204, toolbar 228, camera view 224, and user interface wrapper 220. The aforementioned elements may include all the features previously described, in addition to the modifications and/or additions described herein.

In some examples, the user interface wrapper 220 may include content that may be relevant to the healthcare provider such as, but not limited to, patient information 236, patient history (not shown), content library 240, preliminary assessment 244, and question recommendations 248. User interface wrapper 220 associated with the second user device (i.e., the user device operated by the healthcare provider) may not include certain elements, including, but not limited to, advertisements, a word cloud (e.g., key phrase panel 212 described in FIG. 2A), and/or healthcare provider information (e.g., healthcare provider information 216 described in FIG. 2A).

Patient information 236 may display a high-level summary of the patient for reference by the healthcare provider. The data displayed in patient information 236 may be received from the medical history of the patient, intake paperwork, user input (e.g., from the patient, healthcare provider, etc.), any combination thereof, or the like. Patient information 236 may include the patient's name, gender/sex, age, weight, relevant ailment, information that may assist the healthcare provider over the duration of the communication session, any combination thereof, or the like. The data presented in patient information 236 may be selected by the healthcare provider, an individual directed by the healthcare provider, the associated health system, a machine-learning algorithm, any combination thereof, or the like. The data presented in patient information 236 may be unique to the communication session. During a subsequent communication session, patient information 236 may include different information based on the patient and the subsequent communication session. Patient information 236 may also comprises a link for the user device to select and/or view documents related to the patient, such as a medical history, submitted photos, video (if the first user device is transmitting video), contact information, insurance information, any combination thereof, or the like. In some examples, the communication network may provision a first machine-learning model capable of conducting facial analysis to supplement information provided in patient information 236. The video stream of the patient shown via camera view 224 on the second user device may be received by the first machine-learning model. The first machine-learning model may conduct facial analysis of the patient and infer one or more characteristics about the patient, including pain, confusion, interest, contentment, anger, frustration, sadness, etc.

Content library 240 may be a storage location accessible by the second user device via the telehealth environment, communication session environment, provider interface 232, any combination thereof, or the like. Content library 240 may contain content selected by the healthcare provider that can be selected, accessed, or presented by the second user device. In some instances, content library 240 may be access restricted to prevent access to data stored in content library 240 by users other than the healthcare provider. The storage location of content library 240 may be a local storage device, cloud storage, shared network drive, any combination thereof, or the like. Content library 240 may be store content associated with a particular communication session or associated with a particular patient. For example, content library 240 be store different content during different communication session involving a same patient and healthcare provider.

The second user device may select content (e.g., data, information, files, etc.) to be stored in content library 240 and display selected content from content library 240 through collaborative window 204. Data can be stored in the storage location before the communication session, during the communication session, and/or after the communication session. The second user device may select data for storage in a local storage medium (e.g., local storage device, cloud storage, shared network drive, any combination thereof, or the like), data stored in a remote storage medium or network (e.g., cloud network, webpage, storage area network, database, server), data from collaborative window 204 (e.g., data provide by the patient or by the healthcare provider), or the like for storage in content library 240. For example, content library 240 may include presentations, informative videos, diagrams, charts, tables, photos, scans, test results, and/or any content that may be relevant to the communication session. In some instances, content library 240 may include data generated by a machine-learning algorithm (e.g., such as a large language model, classifier, etc.) trained to identify relevant content for the communication session according to a number of factors, including, but not limited to, patient data, medical history, communication session history, healthcare provider practice area, the communication session, any combination thereof, or the like. In some examples, the machine-learning algorithm may dynamically generate recommendations within content library 240 that pertain to the communication session at a given point. In some examples, content library 240 may be searched with one or more natural language commands from the healthcare provider. A machine-learning model trained to interpret natural language may receive the one or more natural language commands and output relevant data. For example, the healthcare provider may request, “show me the results of the patient's bloodwork,” and a second machine-learning model may receive the request, query the storage location, and output the most recent bloodwork results associated with the patient.

Preliminary assessment 244 may contain relevant data associated with the patient. Preliminary assessment 244 may display one or more physical characteristics of the patient (e.g., height, weight, etc.), lifestyle habits of the patient (e.g., exercise, smoking, drinking, etc.), and/or information related to the reason for the communication session (e.g., injury details, symptoms, etc.). The data may be populated using information obtained during the patient intake process, from forms shared from another healthcare provider (e.g., with consent of the patient), medical history of the patient, generated using a machine-learning algorithm trained to infer additional details about a patient, any combination thereof, or the like. Preliminary assessment 244 may be dynamic and may change throughout the duration of the communication session according to the conversation between the healthcare provider and the patient. Preliminary assessment 244 may add additional data and/or may change existing data (e.g., change weight from 160 lbs. to 165 lbs.).

Question recommendations 248 may generate possible prompts to the second user device. The second user device may present a possible prompt to the first user device (i.e., user device operated by the patient) and/or other user device connected to the communication session. The prompts may be generated by a third machine-learning algorithm trained to aggregate patient data and historical data to articulate pertinent questions. In some examples, the possible prompts may be populated by the healthcare provider, or someone directed by the healthcare provider. The possible prompts may also be generated by any combination of the above. Question recommendations 248 may dynamically change according to the audio transmitted over the duration of the communication session. An NLP system may identify if a possible prompt has been recognized within the transmitted audio (e.g., communicated by a patient or healthcare provider) and may remove it from question recommendations 248. In some instances, the third machine-learning algorithm may prioritize the possible prompts (e.g., putting the most important prompt at the top of the list) and may dynamically alter the prioritization of the possible prompts according to the transmitted audio of the communication session examined by the NLP system. For example, the third machine-learning algorithm may generate the prompt, “do you remember a distinct date or event when the knee pain started?” As the communication session progresses, the third machine-learning algorithm may remove the prompt from the list within question recommendations 248 if the healthcare provider asks the question (or a similar question) or if the patient provides an answer to the question, or the prompt may be moved downward on the list as the conversation between the healthcare provider and the patient gradually moves past introductory conversation and general background information. Alternatively, if the third machine-learning algorithm may move the prompt up the list to ensure the healthcare provider addresses the prompt.

In some examples, the communication network may provision one or more machine-learning models, trained to analyze natural language and aggregate data, to analyze the data exchanged between the first user device and the second user device over the duration of the communication session. This may include audio segments, visual data, data shown in patient information 236, contents of collaborative window 204, any combination thereof, or the like. The one or more machine-learning models may automate one or more tasks associated with the second user device, including, but not limited to, SOAP note generation (e.g., subjective, objective, assessment, and plan note representing a widely used method of documentation for healthcare providers), billing code generation, appointment scheduling, referral to a specialist, prescriptions, scheduling diagnostic tests, clinical decision support, any combination thereof, or the like.

At the conclusion of the communication session, provider interface 232 may automatically close and the communication network may automatically redirect the user device to another location within the communication network or other connected webpage. Content library 240, preliminary assessment 244, question recommendations 248, and/or other content displayed on user interface wrapper 220 may be made accessible to one or more user devices connected to the communication session after the conclusion of the communication session via a transmitted link, a patient portal, the telehealth environment, an application or service, a webpage, any combination thereof, or the like.

FIG. 2C illustrates an example temporary environment of the communication network configured for a particular user device according to aspects of the present disclosure.

Temporary environment 252 may be an interim environment for user devices before a communication session begins or before a particular user device connects to the communication session (e.g., such as the user devices operated by the healthcare provider). For example, a first user device (i.e., user device associated with the patient) may be connected to temporary environment 252 while waiting for a second user device (i.e., user device associated with the healthcare provider) to connect to the communication session. Temporary environment 252 may allow the first user device to complete administrative tasks (e.g., filling out forms, providing information, etc.), access information associated with the communication session or healthcare provider, etc.) while waiting for the second user device to connect to the communication session. Temporary environment 252 may include a user interface comprising one or more elements, including, but not limited to, temporary environment wrapper 268, chatbot interface 264, task list 256, and/or administrative tasks 260. The one or more elements may be selected by connected user devices, the healthcare provider, a machine-learning algorithm, associated health system, any combination thereof, or the like. The one or more elements within temporary environment 252 may be unique to the communication session. The one or more elements may be visual or may contain interactive elements, wherein the first user device may “click” or “select” specific portions to view additional content or where the first user device may provide information to one or more other devices connected to the communication session. In some examples, temporary environment 252 may prompt the first user device to provide remote device data. The remote device data may include, but is not limited to, data from a wearable device (e.g., a sensor-based application such as a smartwatch, smartphone, health tracker data, heart rate monitor, etc.) or data aggregation application (e.g., diet monitoring application, activity monitor, calendar, etc.). The first user device may permit the communication network to access the remote device data or may restrict access to one or more data points, sources, time periods, etc.

Temporary environment wrapper 268 may comprise a graphic overlay that displays content and/or the one or more elements within temporary environment 252. Temporary environment wrapper 268 may include healthcare provider information (e.g., healthcare provider information 216), the associated health system logo, the communication network logo, and/or any element and/or aspect of user interface wrapper 220 of FIG. 2A. The content display on temporary environment wrapper 268 may cycle throughout a time interval corresponding to the time over which the user device is connected to the temporary environment 252.

Task list 256 may include an identification of a set of tasks that may be completed by the user device within the time interval. The tasks of task list 256 may be related to the current communication session, historical communication sessions, identified ailments, insurance information of the patient, a tutorial of temporary environment 252 and/or the communication network, any combination thereof, or the like. Tasks of task list 256 may redirect the user device to an appropriate platform to complete the individual item (e.g., using pointers, hyperlinks, application programming interfaces, remote procedure calls, etc.). The tasks may be generated by the healthcare provider, the associated health system, and associated health clinic, a nurse or other provider, the patient, a machine-learning algorithm, any combination thereof, or the like. Communication network 120 may monitor information received from user devices to determine if information associated with task has been received completing the task. Communication network 120 may automatically remove the task from task list 256. For example, communication network 120 may determine that the user device uploaded a photo, thus a “check mark” will appear next to the appropriate task. The communication may continue before the tasks of task list 256 are completed.

Chatbot 264 may be a virtual chat platform enabled by generative artificial intelligence or another machine-learning algorithm (e.g., large language models, natural language models, etc.). The user device may input a query into chatbot 264, and chatbot 264 may output an appropriate response according to configurable restrictions that can be applied to chatbot 264 (e.g., excluding confidential information, diagnosis, and other medical opinion related information, etc.). The configurable restrictions may be set by the associated health system, the associated health clinic, the healthcare provider, hardcoded, any combination thereof, or the like. Conversations between chatbot 264 and the user device may be saved in local memory and/or may be distributed to the user device using a digital messaging service (e.g., email, text messaging, direct messaging, instant messaging, as a file in the telehealth environment, etc.). Data exchanged between chatbot 264 and the user device may be encrypted and/or secured by another manner to comply with the Health Insurance Portability and Accountability Act (HIPAA), healthcare regulations, privacy policies, and other regulations and/or policies.

Administrative tasks 260 may include an identification of potential administrative actions the user device may complete. The tasks shown in administrative tasks 260 may be related to various tasks pertaining to the patient's medical records, contact information, communication session scheduling, requesting documentation or records, any combination thereof, or the like. A task in administrative tasks 260 may be selected to redirect the user device to an appropriate platform to complete the individual item (e.g., using pointers, hyperlinks, application programming interfaces, remote procedure calls, etc.). The tasks may be generated by the healthcare provider, an associated health system, and associated health clinic, a nurse or other provider, the patient, a machine-learning algorithm, any combination thereof, or the like.

If the first user device is executing operations of temporary environment 252 when and the second user device connects to the communication session, the first user device may receive a notification indicating that the temporary environment 252 will be terminated. In some instances, communication network 120 may automatically redirect the first user device to the communication session. In other instances, the first user device may be presented with a “countdown” to be automatically redirected (e.g., once the user device operated by the healthcare provider connects, the user device has 30 seconds to join the communication session or the user device will be automatically joined). In still yet other instances, communication network 120 may redirect the first user device upon completion of an operation by the first user device (e.g., selecting a “join session” button, scheduling a follow-up session, completing intake forms, etc.). Once temporary environment 252 terminates, user devices may be prevented from accessing temporary environment 252. The one or more elements presented in temporary environment 252 may be accessible in a different environment, such as patient interface 200, telehealth environment, or another interface associated with the patient within the communication network.

FIGS. 2A-2C illustrate various example user interfaces of the communication network. The example user interfaces are merely examples implementations of the contents of this specification. FIGS. 2A-2C are in no way intended to limit the scope of this specification.

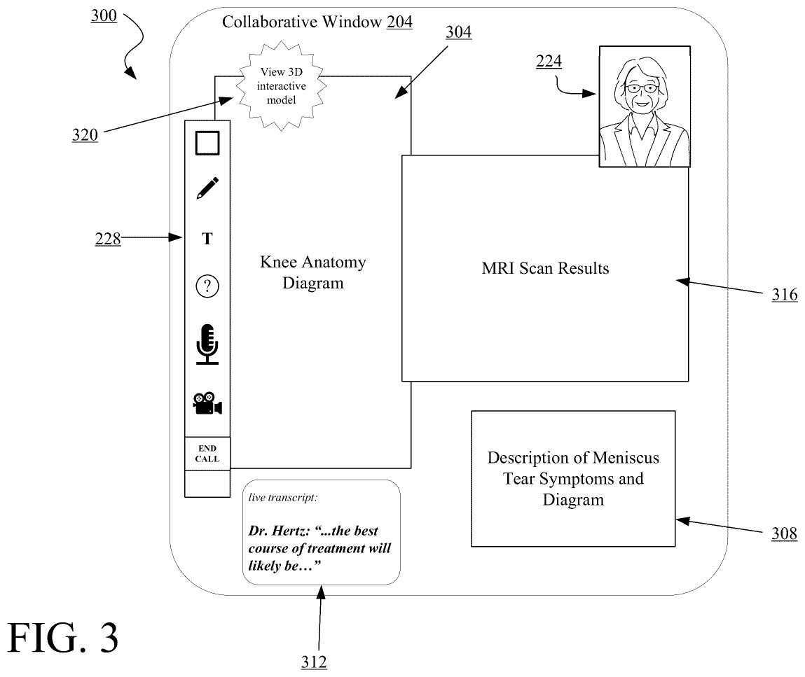

FIG. 3 illustrates an example collaborative window containing AI-generated elements according to aspects of the present disclosure. Window 300 may include a collaborative window (e.g., such as collaborative window 204 of FIG. 2A), which may include any aspect and/or element of collaborative window 204, including toolbar 228 and camera view 224. A first user device (e.g., a user device operated by the patient) and a second user device (e.g., user device operated by the healthcare provider) may share content in the same manner as disclosed in FIGS. 2A-2B.

The communication network may use various machine-learning models to generate AI-assisted objects that can be embedded into a collaborative window. New AI-assisted objects may be generated using one or more machine-learning models (e.g., of machine-learning models 148 of FIG. 1, other machine-learning models, etc.). In some instances, AI-assisted objects may be generated from existing objects of collaborative window 204. For example, AI-assisted objects may be generated by modifying an existing object of collaborative window 204 using the one or more machine-learning models.

The communication network may provision one or more machine-learning models to generate new AI-assisted objects. The one or more machine-learning models may be configured to extend the functionality of the collaborative window with, for example, natural language processing (e.g., such as a large language model, bi-directional transformers, zero/few shot learners, deep neural networks, etc.), content generation (e.g., using large language models, deep neural networks, generative adversarial networks, etc.), single variate or multivariate classifiers (e.g., k-means, k-nearest neighbors, random forest, logarithmic regression, decision trees, support vector machines, gradient decent, etc.), image processing (e.g., using deep neural networks, convolutional neural networks, etc.), sequenced data processors (e.g., such as recurrent neural networks, etc. capable of processing datasets organized according to a taxonomic sequence), and/or the like. For example, the one or more machine-learning models may be configured to process natural language communications (e.g., such as gestures, speech, text, etc.) to provide real-time translations and/or transcriptions, generate natural language communications capable of autonomous communication (e.g., a communication bot, etc.) or content generation (e.g., such generating natural language responses to requests for information, etc.), user authentication (e.g., to ensure users connected to the communication session are authorized to do so), and/or the like.

The communication network may pass communications extracted over the communication session to a processor (e.g., ML core process 132 of FIG. 1) to process the communications using the one or more machine-learning models. The processor may monitor the one or more machine-learning models, train new machine-learning models, retrain (or reinforce) existing machine-learning models, delete machine-learning models, and/or the like. Since the processor manages the operations of a variety of machine-learning models, each request to the processor may include an identification of a particular machine-learning model, a requested output, or the like, to enable the processor to execute an appropriate machine-learning model or instantiate and train a new machine-learning model. Alternatively, the processor may analyze data to be processed that is included in the request to select an appropriate machine-learning model configured to process data of that type.

The processor may execute the one or more machine-learning models upon detecting a change in the communication channel (e.g., a change in video from a user device, an audio segment is transmitted over the communication channel, content is added or removed from a user interface of the communication session (e.g., such as patient interface 200, provider interface 232, temporary environment 252, or any other interface displayed to a user device), content is modified from a user interface of the communication session (e.g., such manipulating an image, etc.), a timestamp relative to a start time of the communication session, etc.). Alternatively, or additionally, the processor may continually execute one or more of the one or more machine-learning models to continually generate corresponding outputs by executing one or more of the one or more machine-learning models in regular time intervals. The processor may evaluate the outputs of the one or more machine-learning models to determine whether to manipulate a user interface of the communication session based on the output (e.g., present automatically generated content, modify word wall weights or words, add/remove suggested questions, initiate an automated conversation with a bot, provide information associated with keywords spoken during the communication session, etc.). For instance, the processor may assign a confidence score to each output from each machine-learning model based on a likelihood that the output from the machine-learning model corresponds to a current context of the communication session or a likelihood that the output would be selected by a user of the communication session for presentation within the collaborative window if presented to the user for selection. The processor may determine to manipulate the user interface when the confidence score is greater than a threshold.

For example, the processor may detect a new audio segment communicated over the communication session. The processor may execute a first machine-learning model (e.g., such as a recurrent neural network) to process the audio segment to identify the words within the audio segment (if any) and a sentiment (e.g., a predicted meaning of the individual words, the words as a whole, the audio segment as a whole, etc.). The processor may execute one or more second machine-learning models (e.g., such as a classifier, a large language model and/or transformer, a generative adversarial network, etc.) to generate content corresponding to the words and/or sentiment that can be embedded within collaborative window 204. For instance, the first machine-learning model may identify the words of a communication as “My knee is swollen and painful” and a sentiment of “symptoms.” The one or more second machine-learning model may process the words and sentiment to generate and/or modify content of collaborative window 204, such as information about ailments associated with knee pain and/or knee swelling, interactive graphics and models of the knee, links to additional information pertaining to knee pain, data regarding particular tests or test results associated with the knee, exercises or treatments for knee pain and/or knee swelling, etc.

For example, the one or more second machine-learning model may generate objects 304-320 and present objects 304-320 within various locations of the collaborative window. Object 304 may include a representation of the anatomy of the knee (e.g., such as a two- or three-dimensional image, an interactive graphic, a video, etc.). In some instances, the machine-learning model may generate the representation of the anatomy of the knee. In other instances, the machine-learning model may retrieve the representation of the anatomy of the knee from a database of annotated, anatomical representations. The machine-learning model may modify the representation by highlighting or shading particular anatomy, adding circles or arrows to point out various features, adding text, adding links, etc. In some instances, the one or more second machine-learning models may add additional versions of object 304. For example, the one or more second machine-learning models may generate a three-dimensional representation of a two-dimensional image and/or an interactive version of the two-dimensional image, etc. The processor may generate object 320 that can be selected (or hovered over) to display alternative versions of object 304).

Object 308 may include additional information associated with the words of the communications. The additional information may be automatically generated (e.g., using an LLM, or the like), retrieved from a database, retrieved from the Internet, etc. In some instances, object 308 may be modified to include additional information (e.g., such as a circles or arrows over relevant features, highlights or color shift of an image, text, any combination thereof, or the like). The additional information may be presented as a graphical overlay when the user selects or hovers over object 308. In some instances, the additional information may include one or more links that may cause a user device to navigate to webpages (e.g., in a separate browser or via a pop-up window) from which the additional information was sourced or to webpages with information associated with the words.

The processor may use machine-learning models to generate objects from test results such as X-rays, magnetic resonance imaging (MRI), computerized tomography (CT) scans, blood work results, etc. For example, processor may use the first machine-learning model and/or the one or more second machine-learning models to generate object 316 that may include a representation of test results. In some examples, the particular test results displayed may be based on a current context of the communication session (e.g., a classification of words identified by the first machine-learning model, keywords in text communicated by a user device, etc.). For example, the first machine-learning model may identify a spoken word such as “MRI,” “scan,” imagery,” etc. that may be classified (e.g., by the first machine-learning model and/or a second machine-learning model of the one or more second machine-learning models) as being associated with MRI test results. The processor may then retrieve the MRI test results from memory. The one or more second machine-learning models may generate object 316 including a representation of the MRI scan. Alternatively, the first user device and/or the second user device may request presentation of particular test results causing the processor to generate object 316 with the requested test results. The representation may be annotated and/or modified by highlighting or shading particular portions of the test results, adding circles or arrows to point out various features, adding text, adding links, converting from a three-dimensional representation to a two-dimensional representation, converting from a two-dimensional representation to a three-dimensional representation, changing a resolution of the representation, zooming in or out of the representation, etc.

In some examples, the processor may generate a real-time transcription of the audio of the communication session. For example, the process may execute a machine-learning model (e.g., a recurrent neural network and/or other deep neural networks) capable of speaker diarization (the process of partitioning an audio stream with multiple people into homogeneous segments associated with each individual), such as the first machine-learning model. The first machine-learning model may receive audio segments from the audio channel of the communication session and generate object 312 including a real-time, visual transcript (e.g., “closed captions”) of the communication session corresponding to the words spoken, audio transmitted, and/or other sounds exchanged over the duration of the communication session. Object 312 may update in real-time over the duration of the communication session. In some examples, the visual transcript displayed via object 312 may be in the same language as is spoken and/or detected by the machine-learning model over the duration of the communication session. In other examples, the processor may train the first machine-learning model to output words in a language spoken by (e.g., as detected by the first machine-learning model) or selected by user of a user device regardless of the language in which the words are initially communicated. For example, if the patient and the healthcare provider speaks English and Spanish, respectively, object 312 may display the English translation of the healthcare provider's speech to the patient and the Spanish translation of the patient's speech to the healthcare provider. In some examples, the machine-learning model may receive audio input corresponding to the communication session and filter out the audio from a particular individual, user device, and/or other source of audio such that the filtered-out audio may not be stored within the communication network. For example, audio input received from the patient may be removed from object 312, the complete transcript of the communication session, and/or other audio recording mechanisms associated with the communication session to comply with HIPPA and/or other related privacy policies.

In some examples, the first machine-learning model may receive video segments from a video channel of the communication session and generate object 312. For example, if a patient communicates with American Sign Language (ASL), a camera directed at the patient may receive input of the patient speaking in ASL, input the video data into the first machine-learning algorithm capable of image processing, and output a visual transcript displayed via object 312 to the healthcare provider. In some other examples, the first machine-learning model may interpret communications via video segments from a video channel via eye-tracking technology. For example, the first machine-learning model may receive video data of a patient and a corresponding visual map and output a visual transcript displayed via object 312 indicating a communication from the patient.

In some examples, the complete transcript, object 312, objects displayed on collaborative window 204 (either selected for sharing by a user device connected to the communication session and/or AI-assisted objects), and any other data stored pertaining to the communication session may be output to a document, webpage, file, location in the communication network, memory address, etc. for reference by one or more user devices connected to the communication session. For example, the first user device may download a document containing a complete transcript of the communication session for additional review by the patient and/or a third party. The complete transcript, object 312, objects displayed on collaborative window 204, and any other data stored pertaining to the communication session may be encrypted in a form inaccessible to the communication network. The stored data may be accessible to the first user device, the second user device, and/or another user device connected to the communication session through unique keys, passcodes, any combination thereof, or the like. The first user device and/or the second user device may access the stored data after the conclusion of the communication session. In some examples, the second user device may review the visual transcript of object 312 before the first user device is permitted to download the document containing the complete transcript of the communication session. For example, the healthcare provider may add additional content and/or explanation to conversation conducted and recorded within the complete transcript of the communication session. In some examples, content and/or explanation added to the complete transcript may be distinguished from the original content of the complete transcript (e.g., different font color, size, style, comments, flags, etc.).

In some examples, the processor may generate new objects to display on a collaborative window. The new objects may supplement existing content displayed on the collaborative window. For example, the processor may execute a machine-learning model (e.g., a convolutional neural network) capable of image recognition and image processing. The machine-learning model may receive image input corresponding to images, objects, graphics, tables, flowcharts, etc. displayed on a collaborative window and generate one or more new objects to display on the collaborative window. The processor may execute one or more additional machine-learning models in conjunction with the machine-learning model (e.g., large language model and/or transformer). For example, the second user device may display a diagram displaying knee anatomy and the machine-learning model may generate one or more additional objects pertaining to the diagram, including an arrow pointing to a tendon referenced by the healthcare provider, a link redirecting the user to an interactive three-dimensional model of a knee, color-coding the diagram (e.g., shading areas of concern in red or irrelevant areas in gray), overlaying an MRI scan of the patient onto the diagram, any combination thereof, or the like.

The AI-generated content may be accessible to user devices connected to the communication session after the conclusion of the communication session via a distributed link, encrypted file, website, patient portal, any combination thereof, or the like. User devices connected to the communication session may supplement the AI-generated content or remove it from the collaborative window associated with the user device. For example, if a machine-learning model supplements an existing diagram of a knee by circling or shading a relevant tendon, the second user device may display an MRI scan of the patient demonstrating injury in the relevant tendon, add a comment to the shaded diagram, edit the dimensions of the shaded diagram, any combination thereof, or the like. A user device may disable AI-generated content within the collaborative window.

The machine-learning models incorporated herein may include single or composite models (e.g., made up of homogeneous or heterogeneous model types). For example, a natural language processing machine-learning model may recognize one or more words and/or phrases and may then utilize a large language model to generate an object pertaining to the words and/or phrases. Additionally, the machine-learning models incorporated herein may detect and identify images, graphics, graphs, charts, test results, etc. and may generate one or more relevant objects.

The machine-learning models may be configured to generate and/or modify elements of content, such as adding links, adding or removing graphic elements (e.g., such as text, highlights, shading, boundary boxes, arrows, etc.), cropping content, adding or reducing magnification, increasing/decreasing the resolution of content, etc. The processor may replace content with the corresponding version of modified content automatically. The processor may operate multiple machine-learning models that may be executed in series and/or in parallel to enable any of the aforementioned functionality described herein. The processor may execute any number of machine-learning models in parallel to provide the functionality of a communication session.

The machine-learning models may be configured to process patient information, including, but not limited to, data contained in a user profile associated with the patient, demographic data (e.g., race, gender, ethnicity, etc.), lab/test results, health and medical history (e.g., height, weight, past surgeries, etc.), preferences (e.g., prefers injections over oral medications), allergies, familial status, contact information, insurance information, data associated with prior communication sessions connected to by the user device associated with the individual user profile, healthcare provider information (e.g., primary care physician, OB/GYN, physical therapist, etc.), medical practice history, lifestyle data (e.g., smoking and alcohol frequency, exercise frequency, tendency to remember to take daily medication, tendency to cancel/reschedule/no-show communication sessions and/or other healthcare appointments, stress level, pollution level of living environment, cleanliness, eating habits, etc.), any combination thereof, or the like. The machine-learning models may be trained to process the patient information and generate a provider dashboard that may be presented to the second user device (e.g., before, during, and/or after the communication session). The provider dashboard may contain a summary of the patient information tailored to the communication session. For example, a provider dashboard generated for a communication session between a healthcare provider and a patient with diabetes may contain information about the patient's most recent A1C test results, blood sugar levels/trends, weight trends, a food journal kept by the patient, etc. In another example, a provider dashboard generated for a communication session between a healthcare provider and a patient with a knee injury may contain MRI scan results, a description of the events surrounding the injury, images of the patient's knee, results of lab work conducted, etc. The provider dashboard may be configured to provide a summary of the most recent information associated with the patient to reduce the time needed for the healthcare provider to prepare for the communication session.

In some examples, the provider dashboard may be available to the healthcare provider prior to an in-person appointment via the communication network. The machine-learning models may be configured to process stored communication sessions, including, but not limited to, transcripts, images, audio, video, and any objects and/or content described in FIGS. 2A-3. The machine-learning models may be trained to process data associated with the communication session, objects of a collaborative window the first user device, the second user device, one or more additional user devices, any combination thereof, or the like to derive datasets usable to modify the communication network and/or subsequent communication sessions. For example, the machine-learning models may identify trends (e.g., based on geographical location, injuries, patient demographics, etc.), define metrics for healthcare providers (e.g., effectiveness, quality of care, diagnosis accuracy, treatment efficacy, efficiency, etc.), identify opportunities for providing products or services to patients or healthcare providers (e.g., identify particular healthcare providers and/or patients that may use or be interesting functions, content, features, products, etc. of the communication network, healthcare providers, healthcare system, etc.), identify a time within a communication session or post-session environment for presenting or providing particular content (e.g., such as content, features, functions, services, etc.), identify healthcare provider pathways and guidelines (e.g., generate one or more treatment steps associated with a disease and a particular class of patients, etc.), generate metadata including additional data associated with the patient and/or the healthcare provider derived from the communication sessions, derive a patient or healthcare provider satisfaction, combinations thereof, or the like. For example, a machine-learning model may process a communication session and generate a manufacturer's coupon applicable for a medication discussed during the communication session and may present the manufacturer's coupon to the patient. As another example, a machine-learning model may identify outcomes of a particular disease and utilize the characteristics of associated patients to define a series of guidelines associated with a particular class of patients. The output from the one or more machine-learning models may be used to modify the communication network and/or the communication session by, for example, modifying graphical user interfaces (e.g., add, removing, rearranging any of objects, icons, features, graphics, etc.; modifying colors, shapes, sizes, resolutions of components of the graphical user interfaces; combinations thereof, or the like), modifying content presented to the first user device and/or the second user device such as the processes or machine-learning models that generate the content, adding or removing functionality, selecting healthcare providers for communication sessions with particular patients, etc.

In some instances, the communication network may be configured to generate recommendations for treatments for the patient based on the communication session and/or a diagnosis provided by the healthcare provider. In some instances, the recommend treatment may be a digital therapeutic (e.g., a device and/or software application configured to monitor patient activity and/or provide various treatments or therapies). Software-based digital therapeutics may be stored in a repository accessible to the communication network. Upon being recommended by the healthcare provider, a software-based digital therapeutic may be pushed to the first user device or another device associated with the patient. Pushing the software-based digital therapeutic may include provisioning the first user device, transmitting the software-based digital therapeutic to the first user device, etc. The software-based digital therapeutic may be configured to monitor the patient and/or provide data to the healthcare provider over a time interval (e.g., in real time or in batches). Hardware-based digital therapeutics may be provided the patient via an in-office healthcare session or via mail.

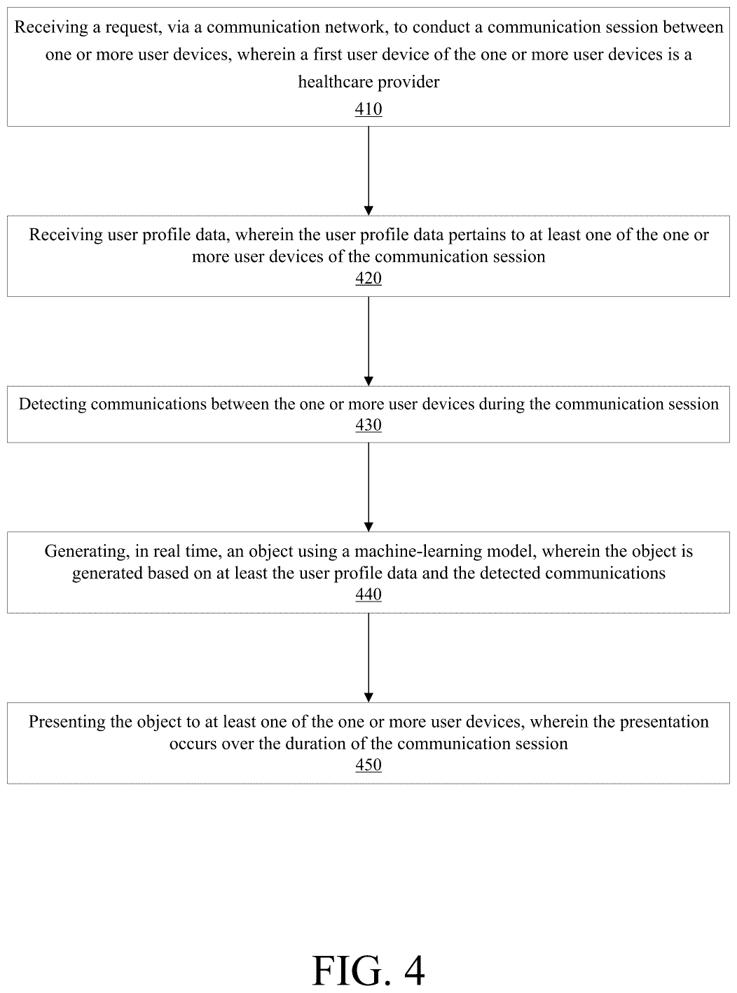

FIG. 4 illustrates a flowchart of an example process for dynamically generating objects within real time collaborative windows according to aspects of the present disclosure. At block 410, a computing device may receive a request to conduct a communication session between one or more user devices. The computing device may be a component (e.g., a processing node, device, etc.) of a communication network (e.g., such as communication network 120 of FIG. 1, or the like). A first user device of the one or more user devices may be operated by a patient and a second user device of the one or more user devices may be operated by a healthcare provider. The computing device may be a processing device (e.g., such as a computer, server, etc.) or virtual environment (e.g., such as a virtual machine, etc.) configured to facilitate any of the features of the communication network described herein.

The first user device may transmit a communication session request to the communication network to request a communication session including the first user device and the second user device. The communication session may be facilitated over one or more communication channels (e.g., telephone or other audio channel, video or other video channel, text such as text messaging or instant messaging, combinations thereof, or the like). In some instances, the communication channel may be asynchronous or communications may be transmitted over the communication channel asynchronously. For example, the first user device may communicate over an audio channel and lack a capability of receiving or processing data received over a video channel (e.g., due to permissions or other security settings, processing capabilities, available resources, available bandwidth, etc.). The second user device may communicate over both audio and video, etc. such that the first user device may receive audio from the second user device and the second user device may receive both audio and video from the first user device.

The communication session request may include a set of communication session parameters. The set of communication session parameters may include, but are not limited to, a quantity of user devices that are authorized to connect to the communication session, an identification of the user devices or the users thereof (e.g., such a device identifier, Internet Protocol address, email address, phone number, username, a user identifier, combinations thereof, or the like), a length of the communication session, an identification of one or more communication channels authorized for the communication session (e.g., such as, audio, video, audiovisual, text messaging, email, instant messaging, combinations thereof, or the like), video settings, sound settings, collaborative environment parameters, privacy and/or encryption parameters, artificial intelligence accessibility, date and time, etc. The set of communication session parameters may be modified at any time prior to and/or during the communication session.

The communication network may transmit a notification to the one or more user devices invited to the communication session using information of the set of communication session parameters. The notification may include a representation of one or more communication session parameters of the set of communication session parameters. The one or more user devices may accept or decline the communication session. A user device of the one or more second user devices may request a modification to one or more of the communication session parameters (e.g., such the date/time of the communication session, identification of user devices authorized to access the communication session, etc.). The communication network may receive a response from the one or more user devices (or user devices associated with the communication request). The one or more user devices may modify a response of accepting or declining the communication session request (e.g., changing an acceptance to a decline, changing a decline to an acceptance, etc.). The communication session parameters may determine an action if no response is received from a user device of the one or more user devices. In some instances, the communication network may indicate that a lack of response from the user device may be an acceptance. In other instances, the communication network may indicate that a lack of response from the user device may be a decline.

At block 420, the computing device may receive user profile data. The user profile data may correspond to at least one of the one or more user devices of the communication session. User devices connected to the communication session may be associated with individual user profiles stored within, or in association with, the computing device. The individual user profiles may be completed and stored prior to the communication session. The user profile data within the individual user profiles may include, but is not limited to, demographic data (e.g., race, gender, ethnicity, etc.), health and medical history (e.g., height, weight, past surgeries, etc.), preferences (e.g., prefers injections over oral medications), allergies, familial status, contact information, insurance information, data associated with prior communication sessions connected to by the user device associated with the individual user profile, healthcare provider information (e.g., primary care physician, OB/GYN, physical therapist, etc.), medical practice history, lifestyle data (e.g., smoking and alcohol frequency, exercise frequency, tendency to remember to take daily medication, tendency to cancel/reschedule/no-show communication sessions and/or other healthcare appointments, stress level, pollution level of living environment, cleanliness, eating habits, etc.), any combination thereof, or the like.

At block 430, the computing device may detect communications between the one or more user devices over a duration of the communication session. The communication may be transmitted any device of the one or more user devices connected to the communication session. The communication may include an audio segment extracted from an audio channel of the communication session (e.g., a conversation between users of the first user device and the second user device), gestures (e.g., sign language, non-verbal communications, etc.), text, combinations thereof, or the like). In some instances, the communication may include, or is associated with, shared content embedded within a collaborative window (e.g., such as collaborative window 204 as previously described) and visible and/or accessible to at least one user device connected to the communication session. The communication may be related to data from the user profile data, such as medical history, reason for the initiation of the communication session, lifestyle information, any combination thereof, or the like. The communication may be interpreted by a machine-learning algorithm, natural language processing machine-learning model, etc. by converting the communication into a neutral format (e.g., alphanumeric text using one or more machine-learning model such as a speech-to-text model, etc.) and classifying the neutral format (e.g., ushing the one or more machine-learning models or other machine-learning models).

At block 440, the computing device may generate, in real time, an object using a machine-learning model. The object may be generated based on the user profile data and the detected communications. The object may be configured to be embedded within a graphical user interface (e.g., such as a collaborative window, etc.) for presentation to the one or more users connected to the communication session. The object may be a graphical user interface object (e.g., such a static, dynamic, and/or interactive image, graph, video, graphic, etc.), a data object (e.g., providing data to one or more other objects of the graphical user interface, functional objects (e.g., one or more executable functions that extend a functionality of the graphical user interface and that can be separate from or embedded within one or more other objects of the graphical user interface), combinations thereof, or the like.

For example, the object may be a real-time, visual transcript (e.g., object 312 of FIG. 3) that presents an alphanumeric representation of words spoken via the audio channel of the communication channel. The machine-learning model may be configured to convert an audio segment to alphanumeric text. The visual transcript may include a complete visual record of the audio-channel. For example, the object may include a scroll bar or other mechanism to allow a user to review an alphanumeric representation of any audio segment transmitted over the audio channel. The visual transcript may include an indication as to which user device and/or the user thereof transmitted a particular audio segment. In some instances, the visual transcript may be filtered by removing alphanumeric representations that are superfluous (e.g., such as interjections, disfluencies, etc.) or not relevant to a purpose of the communication session.

Examples of other objects include, but are not limited to, graphics (e.g., such as graphic 304 of FIG. 3 including images, two or three dimensional models, graphs, videos, etc.), custom generated or curated information (e.g., such as object 308 of FIG. 3 including automatically generated text or other informational content contextually relevant to communications transmitted over the communication channel such as descriptions, definitions, summaries, etc.), test results (e.g., such as object 316 of FIG. 3), a visual indicators of interactive content (e.g., such as object 320 of FIG. 3 such as a selectable objects configured to modify the graphical user interface, modify objects therein, link to webpages or other information sources, etc.).

At block 450, the computing device may present the object to at least one of the one or more user devices, wherein the presentation occurs over the duration of the communication session. The object may be displayed on a collaborative window synchronized in real-time across user devices connected to the communication session (e.g., collaborative window 204 in FIGS. 2A, 2B, and 3). In some examples, the object may be made accessible to the at least one of the one or more user devices after the conclusion of the communication session via a transmitted link, a patient portal, the telehealth environment, an application or service, a webpage, any combination thereof, or the like.

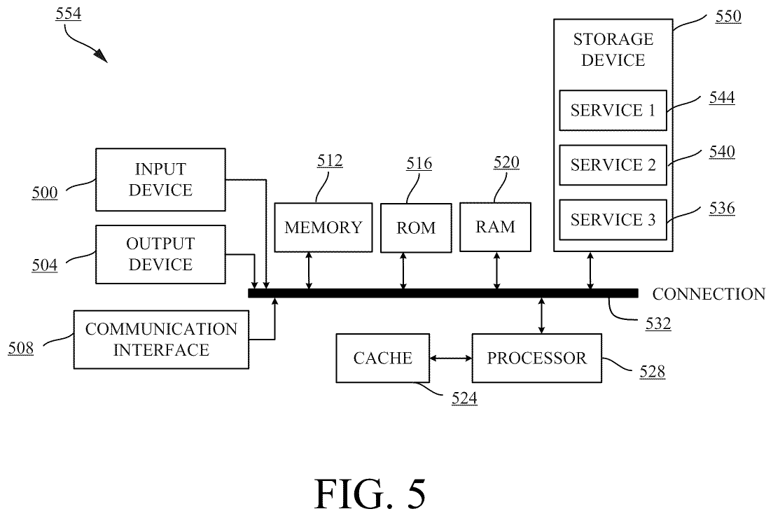

FIG. 5 illustrates an example computing device according to aspects of the present disclosure. For example, computing device 500 can implement any of the systems or methods described herein. In some instances, computing device 500 may be a component of or included within a media device. The components of computing device 500 are shown in electrical communication with each other using connection 506, such as a bus. The example computing device architecture 500 includes a processor (e.g., CPU, processor, or the like) 504 and connection 506 (e.g., such as a bus, or the like) that is configured to couple components of computing device 500 such as, but not limited to, memory 520, read only memory (ROM) 518, random access memory (RAM) 516, and/or storage device 508, to processing unit 510.

Computing device 500 can include a cache 502 of high-speed memory connected directly with, in close proximity to, or integrated within processor 504. Computing device 500 can copy data from memory 520 and/or storage device 508 to cache 502 for quicker access by processor 504. In this way, cache 502 may provide a performance boost that avoids delays while processor 504 waits for data. Alternatively, processor 504 may access data directly from memory 520, ROM 517, RAM 516, and/or storage device 508. Memory 520 can include multiple types of homogenous or heterogeneous memory (e.g., such as, but not limited to, magnetic, optical, solid-state, etc.).

Storage device 508 may include one or more non-transitory computer-readable media such as volatile and/or non-volatile memories. A non-transitory computer-readable medium can store instructions and/or data accessible by computing device 500. Non-transitory computer-readable media can include, but is not limited to magnetic cassettes, hard-disk drives (HDD), flash memory, solid state memory devices, digital versatile disks, cartridges, compact discs, random access memories (RAMs) 525, read only memory (ROM) 520, combinations thereof, or the like.

Storage device 508, may store one or more services, such as service 1 510, service 2 512, and service 3 514, that are executable by processor 504 and/or other electronic hardware. The one or more services include instructions executable by processor 504 to: perform operations such as any of the techniques, steps, processes, blocks, and/or operations described herein; control the operations of a device in communication with computing device 500; control the operations of processing unit 510 and/or any special-purpose processors; combinations therefor; or the like. Processor 504 may be a system on a chip (SOC) that includes one or more cores or processors, a bus, memories, clock, memory controller, cache, other processor components, and/or the like. A multi-core processor may be symmetric or asymmetric.

Computing device 500 may include one or more input devices 522 that may represent any number of input mechanisms, such as a microphone, a touch-sensitive screen for graphical input, keyboard, mouse, motion input, speech, media devices, sensors, combinations thereof, or the like. Computing device 500 may include one or more output devices 524 that output data to a user. Such output devices 524 may include, but are not limited to, a media device, projector, television, speakers, combinations thereof, or the like. In some instances, multimodal computing devices can enable a user to provide multiple types of input to communicate with computing device 500. Communications interface 526 may be configured to manage user input and computing device output. Communications interface 526 may also be configured to managing communications with remote devices (e.g., establishing connection, receiving/transmitting communications, etc.) over one or more communication protocols and/or over one or more communication media (e.g., wired, wireless, etc.).

Computing device 500 is not limited to the components as shown in FIG. 5. Computing device 500 may include other components not shown and/or components shown may be omitted.

As used below, any reference to a series of examples is to be understood as a reference to each of those examples disjunctively (e.g., “Examples 1-4” is to be understood as “Examples 1, 2, 3, or 4”).

Example 1 is a computer-implemented method, comprising: receiving a request, via a communication network, to conduct a communication session between one or more user devices, wherein a first user device of the one or more user devices is a healthcare provider; receiving user profile data, wherein the user profile data pertains to at least one of the one or more user devices of the communication session; detecting communications between the one or more user devices over a duration of the communication session; generating, in real time, an object using a machine-learning model, wherein the object is generated based on at least the user profile data and the detected communications; and presenting the object to at least one of the one or more user devices, wherein the presentation occurs over the duration of the communication session.

Example 2 is the computer-implemented method of example(s) 1, wherein the object is encompassed in a primary interactive object that is currently presented to at least one of the one or more user devices, whereby the object does not visually display additional content without interaction via the communication network.

Example 3 is the computer-implemented method of example(s) 1-2, wherein the object encompasses the entirety of a visual virtual environment on a user device of the one or more user devices, and wherein the object contains one or more interactive elements.

Example 4 is the computer-implemented method of example(s) 1-3, wherein the object includes a visual transcription of an audio channel of the communication session.

Example 5 is the computer-implemented method of example(s) 1-4, wherein the machine-learning model generates the object based on prior communication sessions between at least one of the one or more user devices.

Example 6 is the computer-implemented method of example(s) 1-5, wherein the object incorporates data from the communication session and/or prior communication sessions to generate updated clinical guidelines.

Example 7 is the computer-implemented method of example(s) 1-6, wherein the communication session includes an audio channel and a video channel.

Example 8 is the computer-implemented method of example(s) 1-7, wherein a video channel associated with the communication session is configured to present a collaborative window in place of a representation of a user of a user device.

Example 9 is the computer-implemented method of example(s) 1-8, wherein a recording of the communication session contains consolidated video content from the one or more user devices.

Example 10 is the computer-implemented method of example(s) 1-9, wherein the user profile data is generated by at least one of a machine-learning algorithm, prior communication sessions, and data gathered prior to the communication session.

Example 11 is a system comprising of one or more processor and a non-transitory computer-readable medium storing instructions that when executed by the one or more processors cause the one or more processors to perform the methods of any of example(s) 1-10.

Example 12 is a non-transitory computer-readable medium storing instructions that when executed by the one or more processors cause the one or more processors to perform the methods of any of example(s) 1-10.

The term “computer-readable medium” includes, but is not limited to, portable or non-portable storage devices, optical storage devices, and various other mediums capable of storing, containing, or carrying instruction(s) and/or data. A computer-readable medium may include a non-transitory medium in which data can be stored in a form that excludes carrier waves and/or electronic signals. Examples of a non-transitory medium may include, but are not limited to, a magnetic disk or tape, optical storage media such as compact disk (CD) or digital versatile disk (DVD), flash memory, memory, or memory devices. A computer-readable medium may have stored thereon code and/or machine-executable instructions that may represent a procedure, a function, a subprogram, a program, a routine, a subroutine, a module, a software package, a class, or any combination of instructions, data structures, or program statements. A code segment may be coupled to another code segment or a hardware circuit by passing and/or receiving information, data, arguments, parameters, or memory contents. Information, arguments, parameters, data, etc. may be passed, forwarded, or transmitted via any suitable means including memory sharing, message passing, token passing, network transmission, or the like.

Some portions of this description describe examples in terms of algorithms and symbolic representations of operations on information. These operations, while described functionally, computationally, or logically, may be implemented by computer programs or equivalent electrical circuits, microcode, or the like. Furthermore, arrangements of operations may be referred to as modules, without loss of generality. The described operations and their associated modules may be embodied in software, firmware, hardware, or any combinations thereof.

Any of the steps, operations, or processes described herein may be performed or implemented with one or more hardware or software modules, alone or in combination with other devices. In some examples, a software module can be implemented with a computer-readable medium storing computer program code, which can be executed by a processor for performing any or all of the steps, operations, or processes described.

Some examples may relate to an apparatus or system for performing any or all of the steps, operations, or processes described. The apparatus or system may be specially constructed for the required purposes, and/or it may comprise a general-purpose computing device selectively activated or reconfigured by a computer program stored in memory of computing device. The memory may be or include a non-transitory, tangible computer readable storage medium, or any type of media suitable for storing electronic instructions, which may be coupled to a bus. Furthermore, any computing systems referred to in the specification may include a single processor or multiple processors.

While the present subject matter has been described in detail with respect to specific examples, it will be appreciated that those skilled in the art, upon attaining an understanding of the foregoing, may readily produce alterations to, variations of, and equivalents to such embodiments. Numerous specific details are set forth herein to provide a thorough understanding of the claimed subject matter. However, those skilled in the art will understand that the claimed subject matter may be practiced without these specific details. In other instances, methods, apparatuses, or systems that would be known by one of ordinary skill have not been described in detail so as not to obscure claimed subject matter. Accordingly, the present disclosure has been presented for purposes of example rather than limitation, and does not preclude the inclusion of such modifications, variations, and/or additions to the present subject matter as would be readily apparent to one of ordinary skill in the art.

For clarity of explanation, in some instances the present disclosure may be presented as including individual functional blocks including functional blocks comprising devices, device components, steps or routines in a method embodied in software, or combinations of hardware and software. Additional functional blocks may be used other than those shown in the figures and/or described herein. For example, circuits, systems, networks, processes, and other components may be shown as components in block diagram form in order not to obscure the embodiments in unnecessary detail. In other instances, well-known circuits, processes, algorithms, structures, and techniques may be shown without unnecessary detail in order to avoid obscuring the embodiments.

Individual examples may be described herein as a process or method which may be depicted as a flowchart, a flow diagram, a data flow diagram, a structure diagram, or a block diagram. Although a flowchart may describe the operations as a sequential process, many of the operations can be performed in parallel or concurrently. In addition, the order of the operations may be re-arranged. A process is terminated when its operations are completed but may have additional steps not shown. A process may correspond to a method, a function, a procedure, a subroutine, a subprogram, etc. When a process corresponds to a function, its termination can correspond to a return of the function to the calling function or the main function.

Processes and methods according to the above-described examples can be implemented using computer-executable instructions that are stored or otherwise available from computer-readable media. Such instructions can include, for example, instructions and data which cause or otherwise configure a general-purpose computer, special purpose computer, or a processing device to perform a certain function or group of functions. Portions of computer resources used can be accessible over a network. The computer executable instructions may be, for example, binaries, intermediate format instructions such as assembly language, firmware, source code, etc.

Devices implementing the methods and systems described herein can include hardware, software, firmware, middleware, microcode, hardware description languages, or any combination thereof, and can take any of a variety of form factors. When implemented in software, firmware, middleware, or microcode, the program code or code segments to perform the necessary tasks (e.g., a computer-program product) may be stored in a computer-readable or machine-readable medium. The program code may be executed by a processor, which may include one or more processors, such as, but not limited to, one or more digital signal processors (DSPs), general purpose microprocessors, an application specific integrated circuits (ASICs), field programmable logic arrays (FPGAs), or other equivalent integrated or discrete logic circuitry. Such a processor may be configured to perform any of the techniques described in this disclosure. A processor may be a microprocessor; conventional processor, controller, microcontroller, state machine, or the like. A processor may also be implemented as a combination of computing components (e.g., a combination of a DSP and a microprocessor, a plurality of microprocessors, one or more microprocessors in conjunction with a DSP core, or any other such configuration). Accordingly, the term “processor,” as used herein may refer to any of the foregoing structure, any combination of the foregoing structure, or any other structure or apparatus suitable for implementation of the techniques described herein. Functionality described herein also can be embodied in peripherals or add-in cards. Such functionality can also be implemented on a circuit board among different chips or different processes executing in a single device, by way of further example.

In the foregoing description, aspects of the disclosure are described with reference to specific examples thereof, but those skilled in the art will recognize that the disclosure is not limited thereto. Thus, while illustrative examples of the disclosure have been described in detail herein, it is to be understood that the inventive concepts may be otherwise variously embodied and employed, and that the appended claims are intended to be construed to include such variations. Various features and aspects of the above-described disclosure may be used individually or in any combination. Further, examples can be utilized in any number of environments and applications beyond those described herein without departing from the broader spirit and scope of the disclosure. The disclosure and figures are, accordingly, to be regarded as illustrative rather than restrictive.

The various illustrative logical blocks, modules, circuits, and algorithm steps described in connection with the embodiments disclosed herein may be implemented as electronic hardware, computer software, firmware, or combinations thereof. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present application.

Unless specifically stated otherwise, it is appreciated that throughout this specification discussions utilizing terms such as “processing,” “computing,” “calculating,” “determining,” and “identifying” or the like refer to actions or processes of a computing device, such as one or more computers or a similar electronic computing device or devices, that manipulate or transform data represented as physical electronic or magnetic quantities within memories, registers, or other information storage devices, transmission devices, or media devices of the computing platform. The use of “adapted to” or “configured to” herein is meant as open and inclusive language that does not foreclose devices adapted to or configured to perform additional tasks or steps. Additionally, the use of “based on” is meant to be open and inclusive, in that a process, step, calculation, or other action “based on” one or more recited conditions or values may, in practice, be based on additional conditions or values beyond those recited. Headings, lists, and numbering included herein are for case of explanation only and are not meant to be limiting.

The foregoing detailed description of the technology has been presented for purposes of illustration and description. It is not intended to be exhaustive or to limit the technology to the precise form disclosed. Many modifications and variations are possible in light of the above teaching. The described embodiments were chosen in order to best explain the principles of the technology, its practical application, and to enable others skilled in the art to utilize the technology in various embodiments and with various modifications as are suited to the particular use contemplated. It is intended that the scope of the technology be defined by the claim.

1. A computer-implemented method, comprising:
receiving a request, via a communication network, to conduct a communication session between one or more user devices, wherein a first user device of the one or more user devices is a healthcare provider;
receiving user profile data, wherein the user profile data pertains to at least one of the one or more user devices of the communication session;
detecting communications between the one or more user devices over a duration of the communication session;
generating, in real time, an object using a machine-learning model, wherein the object is generated based on at least the user profile data and the detected communications; and
presenting the object to at least one of the one or more user devices, wherein the presentation occurs over the duration of the communication session.

2. The computer-implemented method of claim 1, wherein the object is encompassed in a primary interactive object that is currently presented to at least one of the one or more user devices, whereby the object does not visually display additional content without interaction via the communication network.

3. The computer-implemented method of claim 1, wherein the object encompasses the entirety of a visual virtual environment on a user device of the one or more user devices, and wherein the object contains one or more interactive elements.

4. The computer-implemented method of claim 1, wherein the object includes a visual transcription of an audio channel of the communication session.

5. The computer-implemented method of claim 1, wherein the machine-learning model generates the object based on prior communication sessions between at least one of the one or more user devices.

6. The computer-implemented method of claim 1, wherein the object incorporates data from the communication session and/or prior communication sessions to generate updated clinical guidelines.

7. The computer-implemented method of claim 1, wherein the communication session includes an audio channel and a video channel.

8. The computer-implemented method of claim 1, wherein a video channel associated with the communication session is configured to present a collaborative window in place of a representation of a user of a user device.

9. The computer-implemented method of claim 1, wherein a recording of the communication session contains consolidated video content from the one or more user devices.

10. The computer-implemented method of claim 1, wherein the user profile data is generated by at least one of a machine-learning algorithm, prior communication sessions, and data gathered prior to the communication session.

11. A system comprising:
one or more processors and a non-transitory computer-readable medium storing instructions that when executed by the one or more processors cause the one or more processors to perform operations that include:
receiving a request, via a communication network, to conduct a communication session between one or more user devices, wherein a first user device of the one or more user devices is a healthcare provider;
receiving user profile data, wherein the user profile data pertains to at least one of the one or more user devices of the communication session;
detecting communications between the one or more user devices over a duration of the communication session;
generating, in real time, an object using a machine-learning model, wherein the object is generated based on at least the user profile data and the detected communications; and
presenting the object to at least one of the one or more user devices, wherein the presentation occurs over the duration of the communication session.

12. The system of claim 11, wherein the object is encompassed in a primary interactive object that is currently presented to at least one of the one or more user devices, whereby the object does not visually display additional content without interaction via the communication network.

13. The system of claim 11, wherein the object encompasses the entirety of a visual virtual environment on a user device of the one or more user devices, and wherein the object contains one or more interactive elements.

14. The system of claim 11, wherein the object includes a visual transcription of an audio channel of the communication session.

15. The system of claim 11, wherein the machine-learning model generates the object based on prior communication sessions between at least one of the one or more user devices.

16. The system of claim 11, wherein the object incorporates data from the communication session and/or prior communication sessions to generate updated clinical guidelines.

17. The system of claim 11, wherein the communication session includes an audio channel and a video channel.

18. The system of claim 11, wherein a video channel associated with the communication session is configured to present a collaborative window in place of a representation of a user of a user device.

19. The system of claim 11, wherein a recording of the communication session contains consolidated video content from the one or more user devices.

20. A non-transitory computer-readable medium storing instructions that when executed by one or more processors cause the one or more processors to perform operations that include:
receiving a request, via a communication network, to conduct a communication session between one or more user devices, wherein a first user device of the one or more user devices is a healthcare provider;
receiving user profile data, wherein the user profile data pertains to at least one of the one or more user devices of the communication session;
detecting communications between the one or more user devices over a duration of the communication session;
generating, in real time, an object using a machine-learning model, wherein the object is generated based on at least the user profile data and the detected communications; and
presenting the object to at least one of the one or more user devices, wherein the presentation occurs over the duration of the communication session.
